(19) United States
(12) Patent Application Publication
(10) Pub. No.: US 2024/0237922 A1
(43) Pub. Date: Jul. 18, 2024

(54) ESTIMATION DEVICE, ESTIMATION SYSTEM, ESTIMATION METHOD, AND RECORDING MEDIUM

(71) Applicant: NEC Corporation, Tokyo (JP)

(72) Inventors: Chenhui HUANG, Tokyo (JP); Fumiyuki Nihey, Tokyo (JP); Hiroshi Kajitani, Tokyo (JP); Kazuki Ihara, Tokyo (JP)

(73) Assignee: NEC Corporation, Tokyo (JP)

(21) Appl. No.: 18/409,016

(22) Filed: Jan. 10, 2024

(30) Foreign Application Priority Data
Jan. 18, 2023 (JP) 2023-006125

(51) Int. Cl. A61B 5/11; A61B 5/00

(52) U.S. Cl. CPC A61B 5/1117; A61B 5/112; A61B 5/6807; A61B 5/7282

(57) ABSTRACT

Provided is an estimation device including a data acquisition unit that acquires first feature amount data related to a physical ability measured according to a gait of a subject and attribute data of the subject, an estimation unit that constructs second feature amount data related to a physical ability factor and an attribute factor by performing principal component analysis on the acquired first feature amount data and the acquired attribute data, and estimates falling risk information according to a falling risk factor using the constructed second feature amount data, and an output unit that outputs the estimated falling risk information.

This application is based upon and claims the benefit of priority from Japanese Patent Application No. 2023-006125, filed on Jan. 18, 2023, the disclosure of which is incorporated herein in its entirety by reference.

### TECHNICAL FIELD

The present disclosure relates to an estimation device, an estimation system, an estimation method, and a recording medium.

### BACKGROUND ART

With the growing interest in healthcare, services for providing information according to a gait have attracted attention. For example, a technique for analyzing a gait using sensor data measured by a sensor mounted on footwear such as shoes has been developed. In time-series data of the sensor data, features of gait events related to a physical condition appear. For example, if information regarding a falling risk can be estimated based on a feature amount extracted from the sensor data, there is a possibility that unexpected falling or the like can be avoided. Elderly people may suffer various injuries due to falls. Therefore, evaluating a falling risk is an important issue in terms of the health of elderly people.

PTL 1 (WO 2021/049196 A1) discloses a factor estimation system that estimates a falling risk factor of a subject. The system according to PTL 1 calculates two or more gait parameters of the subject based on body motion data indicating a body motion of the subject during walking. The system according to PTL 1 estimates one or more principal components included in a falling risk factor of the subject based on the calculated two or more gait parameters.

PTL 2 (JP 2015-202140 A) discloses a system for setting a goal regarding a gait in daily life. The system according to PTL 2 acquires a gait parameter of a certain subject. The system according to PTL 2 calculates a gait feature score from the gait parameter of the subject based on a gait parameter-gait feature score relational expression. The system according to PTL 2 sets a gait feature goal to be accomplished by the subject according to the calculated gait feature score. In addition, the system according to PTL 2 calculates an activity amount from the gait parameter of the subject based on a gait parameter-activity amount relational expression, and sets an activity amount goal to be accomplished by the subject according to the calculated activity amount. PTL 2 discloses that a standardized gait parameter is subjected to principal component analysis, and scores of a plurality of principal components (gait factors) are calculated.

The system according to PTL 1 estimates a falling risk factor of a subject by analyzing a gait condition of the subject based on moving image data captured by a measurement device such as a camera. Therefore, the system according to PTL 1 cannot estimate a falling risk of a subject unless a gait condition can be analyzed based on moving image data.

The system according to PTL 2 measures a gait factor using a measurement device provided therein such as a sheet-type pressure sensor or a motion capture. The system according to PTL 2 cannot measure a gait parameter including a gait factor unless the provided measurement device is used. Therefore, the system according to PTL 2 cannot calculate a gait factor based on a gait in daily life.

An object of the present disclosure is to provide an estimation device and the like capable of estimating a falling risk factor using physical ability-related data measured according to a gait.

### SUMMARY

An estimation device according to an aspect of the present disclosure includes a data acquisition unit that acquires first feature amount data related to a physical ability measured according to a gait of a subject and attribute data of the subject, an estimation unit that constructs second feature amount data related to a physical ability factor and an attribute factor by performing principal component analysis on the acquired first feature amount data and the acquired attribute data, and estimates falling risk information according to a falling risk factor using the constructed second feature amount data, and an output unit that outputs the estimated falling risk information.

An estimation method according to an aspect of the present disclosure includes acquiring first feature amount data related to a physical ability measured according to a gait of a subject and attribute data of the subject, constructing second feature amount data related to a physical ability factor and an attribute factor by performing principal component analysis on the acquired first feature amount data and the acquired attribute data, estimating falling risk information according to a falling risk factor using the constructed second feature amount data, and outputting the estimated falling risk information.

A program according to an aspect of the present disclosure causes a computer to execute acquiring first feature amount data related to a physical ability measured according to a gait of a subject and attribute data of the subject, constructing second feature amount data related to a physical ability factor and an attribute factor by performing principal component analysis on the acquired first feature amount data and the acquired attribute data, estimating falling risk information according to a falling risk factor using the constructed second feature amount data, and outputting the estimated falling risk information.

### BRIEF DESCRIPTION OF THE DRAWINGS

Exemplary features and advantages of the present invention will become apparent from the following detailed description when taken with the accompanying drawings in which:

FIG. 1 is a block diagram illustrating an example of a configuration of an estimation system according to the present disclosure:

FIG. 2 is a block diagram illustrating an example of a configuration of a gait measurement device included in the estimation system according to the present disclosure:

FIG. 3 is a conceptual diagram illustrating an example in which the gait measurement device according to the present disclosure is arranged:

FIG. 4 is a conceptual diagram for explaining an example of a relationship between a local coordinate system and a world coordinate system set in the gait measurement device according to the present disclosure:

FIG. 5 is a conceptual diagram for explaining human body planes used in the description regarding the gait measurement device according to the present disclosure:

FIG. 6 is a conceptual diagram for explaining a gait cycle used in the description regarding the gait measurement device according to the present disclosure;

FIG. 7 is a conceptual diagram for explaining a gait parameter used in the description regarding the gait measurement device according to the present disclosure:

FIG. 8 is a block diagram illustrating an example of a configuration of an estimation device included in the estimation system according to the present disclosure:

FIG. 9 is an example of a falling risk factor used in estimating a falling risk by the estimation system according to the present disclosure:

FIG. 10 is a conceptual diagram illustrating an example in which a physical ability is estimated using a physical ability estimation model by the estimation system according to the present disclosure;

FIG. 11 is a conceptual diagram illustrating an example in which a principal component is estimated through principal component analysis using a feature amount construction model by the estimation system according to the present disclosure;

FIG. 12 is a conceptual diagram illustrating an example in which a falling score is estimated using a falling risk estimation model by the estimation system according to the present disclosure;

FIG. 13 is a table summarizing examples of principal components constructed by performing principal component analysis on indexes with respect to a plurality of subjects classified into two groups depending on whether they have experienced falling:

FIG. 14 is a table summarizing correlation coefficients between an attribute factor or a physical ability factor and principal components constructed by performing principal component analysis on indexes with respect to a plurality of subjects classified into two groups depending on whether they have experienced falling:

FIG. 15 is an ROC curve showing a result of performing 10-split cross validation on a falling risk estimation model constructed by machine learning using a grip strength factor:

FIG. 16 is an ROC curve showing a result of performing 10-split cross validation on a falling risk estimation model constructed by machine learning using an aging factor and a grip strength factor:

FIG. 17 is an ROC curve showing a result of performing 10-split cross validation on a falling risk estimation model constructed by machine learning using an aging factor, a grip strength factor, and a dynamic balance factor;

FIG. 18 is an ROC curve showing a result of performing 10-split cross validation on a falling risk estimation model constructed by machine learning using grip strengths measured in actual physical ability tests:

FIG. 19 is a graph showing a correlation between a falling risk score (true value) estimated by a model constructed using actually measured values (true values) of grip strengths and a falling risk score (estimated value) estimated by a method according to the present example embodiment:

FIG. 20 is a graph showing a frequency distribution of falling risk scores estimated by a falling risk estimation model constructed using grip strengths (true values);

FIG. 21 is a graph showing a frequency distribution of falling risk scores estimated by a falling risk estimation model constructed using grip strengths (estimated values);

FIG. 22 is a flowchart for explaining an example of an operation of the gait measurement device included in the estimation system according to the present disclosure:

FIG. 23 is a flowchart for explaining an example of an operation of the estimation device included in the estimation system according to the present disclosure:

FIG. 24 is a conceptual diagram for explaining an example in which the estimation system according to an example of application of the present disclosure:

FIG. 25 is a conceptual diagram for explaining an example in which the estimation system according to an example of application of the present disclosure:

FIG. 26 is a block diagram illustrating an example of a configuration of an estimation device according to the present disclosure; and

FIG. 27 is a block diagram illustrating an example of a hardware configuration for executing control and processing according to the present disclosure.

### EXAMPLE EMBODIMENT

Example embodiments of the present invention will be described below with reference to the drawings. In the following example embodiments, technically preferable limitations are imposed to carry out the present invention, but the scope of this invention is not limited to the following description. In all drawings used to describe the following example embodiments, the same reference numerals denote similar parts unless otherwise specified. In addition, in the following example embodiments, a repetitive description of similar configurations or arrangements and operations may be omitted.

### First Example Embodiment

First, an estimation system according to a first example embodiment will be described with reference to the drawings. The estimation system according to the present example embodiment measures sensor data according to a gait of a user. The estimation system according to the present example embodiment estimates a factor (a falling risk factor) contributing to a falling risk of the user using the measured sensor data.

In the present example embodiment, an example in which a falling risk is estimated based on the relevance of the falling risk to a feature (also referred to as a gait) included in a gait pattern will be described. In Ir the present example embodiment, the falling risk is estimated using five related items (also referred to as five items) related to a gait and physical attributes of a subject. The five items relate to total body muscle strength (grip strength), dynamic balance, lower limb muscle strength, movement ability, and static balance. These five items correlate to the falling risk. The five items are considered to be relevant to each other to some extent but basically independent of each other. In the present example embodiment, an example in which a falling risk is estimated based on all of the five items will be described.

### (Configuration)

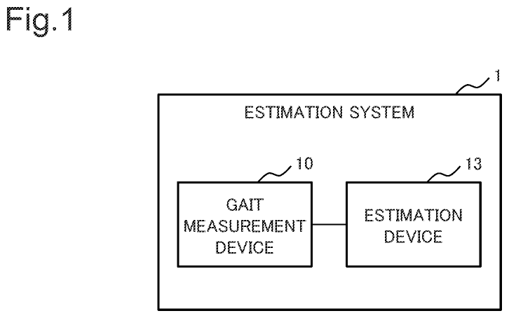

FIG. 1 is a block diagram illustrating an example of a configuration of an estimation system 1 according to the present example embodiment. The estimation system 1 includes a gait measurement device 10 and an estimation device 13. In the present example embodiment, an example in which the gait measurement device 10 and the estimation device 13 are configured as separate pieces of hardware will be described. For example, the gait measurement device 10 is installed on footwear or the like of a subject (a user) who is a target in estimating a falling risk factor. For example, the function of the estimation device 13 is installed in a mobile terminal carried by the subject. Hereinafter, configurations of the gait measurement device 10 and the estimation device 13 will be individually described.

### [Gait Measurement Device]

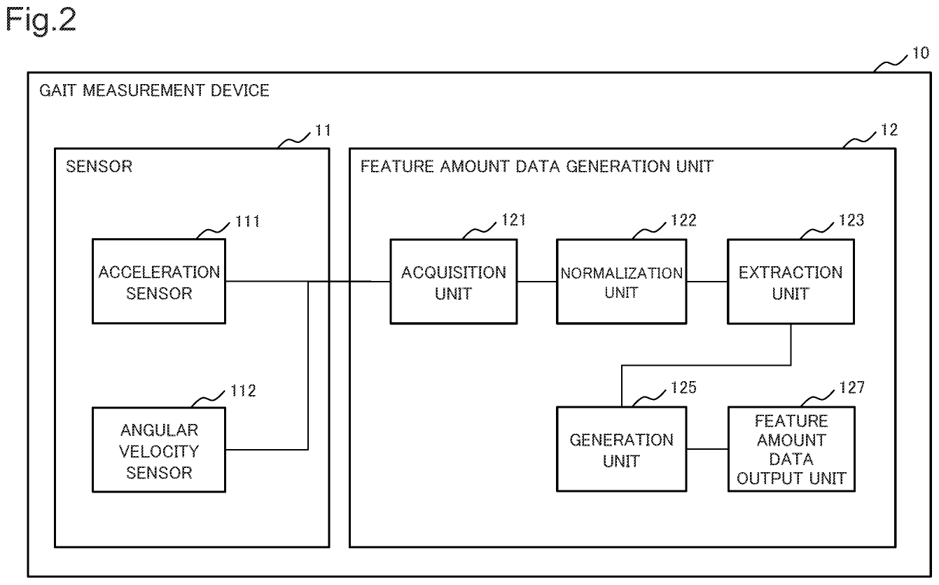

FIG. 2 is a block diagram illustrating an example of a configuration of the gait measurement device 10. The gait measurement device 10 includes a sensor 11 and a feature amount data generation unit 12. In the present example embodiment, an example in which the sensor 11 and the feature amount data generation unit 12 are integrated will be described. The sensor 11 and the feature amount data generation unit 12 may be provided as separate devices.

As illustrated in FIG. 2, the sensor 11 includes an acceleration sensor 111 and an angular velocity sensor 112. In FIG. 2, the acceleration sensor 111 and the angular velocity sensor 112 are included in the sensor 11 as an example. The sensor 11 may include a sensor other than the acceleration sensor 111 and the angular velocity sensor 112. The sensor other than the acceleration sensor 111 and the angular velocity sensor 112 that can be included in the sensor 11 will not be described.

The acceleration sensor 111 is a sensor that measures accelerations in three axial directions (also referred to as a spatial acceleration). The acceleration sensor 111 measures an acceleration (also referred to as a spatial acceleration) as a physical quantity related to a movement of a foot. The acceleration sensor 111 outputs the measured acceleration to the feature amount data generation unit 12. For example, a piezoelectric type sensor, a piezoresistive type sensor, a capacitance type sensor, or the like can be used as the acceleration sensor 111. The measurement method of the sensor used as the acceleration sensor 111 is not limited as long as the sensor can measure an acceleration.

The angular velocity sensor 112 is a sensor that measures angular velocities around three axes (also referred to as a spatial angular velocity). The angular velocity sensor 112 measures an angular velocity (also referred to as a spatial angular velocity) as a physical quantity related to a movement of a foot. The angular velocity sensor 112 outputs the measured angular velocity to the feature amount data generation unit 12. For example, a vibration type sensor, a capacitance type sensor, or the like can be used as the angular velocity sensor 112. The measurement method of the sensor used as the angular velocity sensor 112 is not limited as long as the sensor can measure an angular velocity.

The sensor 11 is achieved by, for example, an inertial measurement device that measures an acceleration and an angular velocity. An example of the inertial measurement device is an inertial measurement unit (IMU). The IMU includes an acceleration sensor 111 that measures accelerations in three axial directions and an angular velocity sensor 112 that measures angular velocities around three axes. The sensor 11 may be achieved by an inertial measurement device such as a vertical gyro (VG) or an attitude heading (AHRS). Furthermore, the sensor 11 may be achieved by a global positioning system/inertial navigation system (GPS/INS). The sensor 11 may be achieved by a device other than the inertial measurement device as long as the sensor can measure a physical quantity related to a movement of the foot.

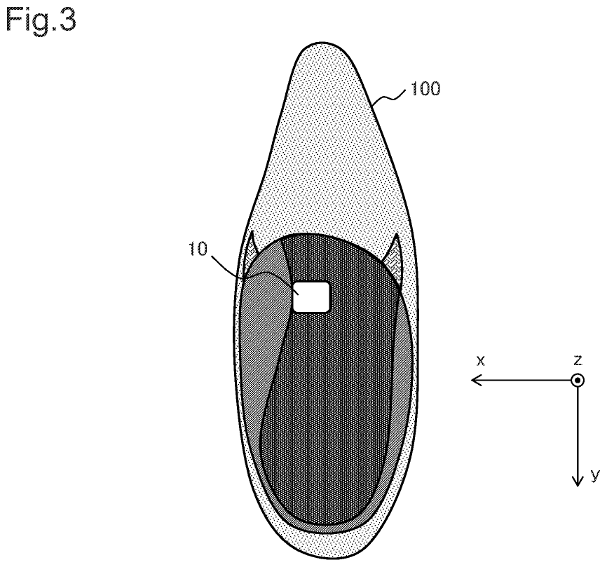

FIG. 3 is a conceptual diagram illustrating an example in which the gait measurement device 10 is arranged in a shoe 100 for a right foot. In the example of FIG. 3, the gait measurement device 10 is installed at a position corresponding to the back side of the arch of foot. For example, the gait measurement device 10 is arranged on an insole inserted into the shoe 100. For example, the gait measurement device 10 may be arranged on the bottom surface of the shoe 100. For example, the gait measurement device 10 may be embedded in the body of the shoe 100. The gait measurement device 10 may be detachable from the shoe 100 or may not be detachable from the shoe 100. The gait measurement device 10 may be installed at a position other than the back side of the arch of foot as long as sensor data related to a movement of the foot can be measured. Furthermore, the gait measurement device 10 may be installed on a sock worn by the user or an accessory such as an anklet worn by the user. Furthermore, the gait measurement device 10 may be directly attached to the foot or may be embedded in the foot. In FIG. 3, an example in which the gait measurement device 10 is installed on the shoe 100 for the right foot. The gait measurement device 10 may be installed on a shoe 100 for a left foot. Furthermore, the gait measurement devices 10 may be installed on shoes 100 of both feet.

In the example of FIG. 3, a local coordinate system including an x axis in the left-right direction, a y axis in the front-back direction, and a z axis in the up-down direction is set with respect to the gait measurement device 10 (the sensor 11). The left side is positive in the x axis, the back side is positive in the y axis, and the upper side is positive in the z axis. The directions of the axes set in the sensors 11 may be the same for the left and right feet, or may be different for the left and right feet. For example, in a case where the sensors 11 produced with the same specifications are arranged inside the left and right shoes 100, the vertical directions (the Z-axis directions) of the sensors 11 arranged on the left and right shoes 100 are the same. In this case, the three axes of the local coordinate system set in the sensor data derived from the left foot and the three axes of the local coordinate system set in the sensor data derived from the right foot are the same on the left and right sides.

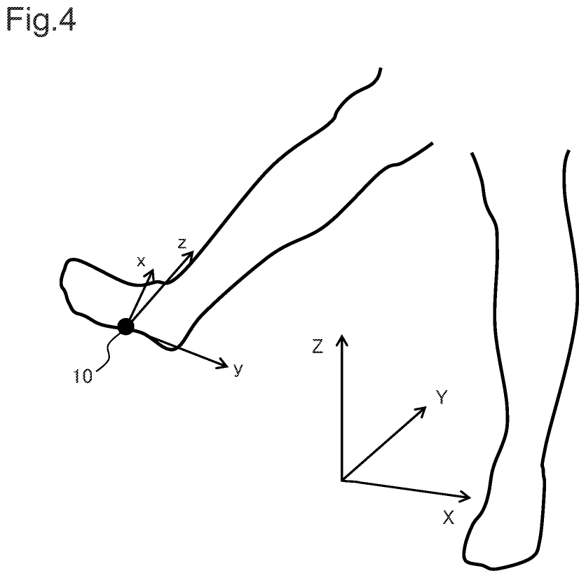

FIG. 4 is a conceptual diagram for explaining a local coordinate system (an x axis, a y axis, and a z axis) set in the gait measurement device 10 (the sensor 11) installed on the back side of the arch of foot and a world coordinate system (an X axis, a Y axis, and a Z axis) set with respect to the ground. In the world coordinate system (the X axis, the Y axis, and the Z axis), in a state where the user facing the moving direction is upright, the lateral direction of the user is set to the X-axis direction (the leftward direction is positive), the back-side direction of the user is set to the Y-axis direction (the backward direction is positive), and the gravity direction is set to the Z-axis direction (the vertically upward direction is positive). Note that, in the example of FIG. 4, although the relationship between the local coordinate system (the x axis, the y axis, and the z axis) and the world coordinate system (the X axis, the Y axis, and the Z axis) is conceptually illustrated, the relationship between the local coordinate system and the world coordinate system, which vary depending on the gait of the user, is not accurately illustrated.

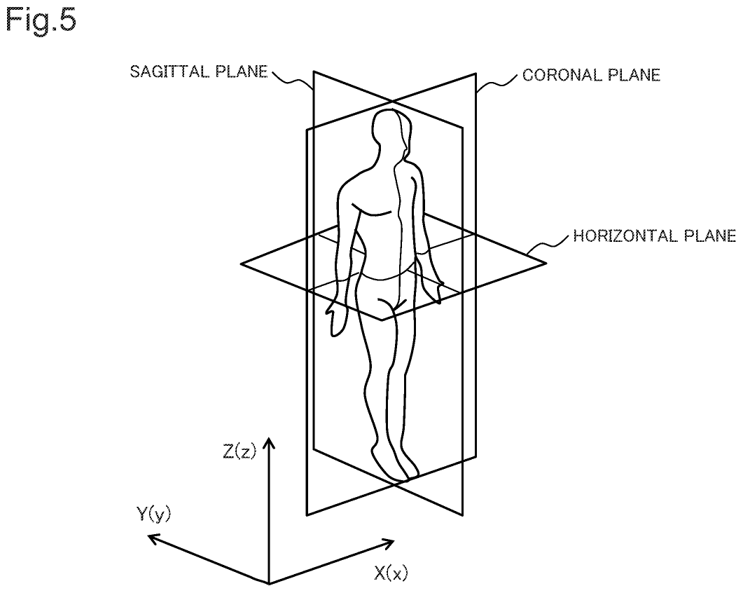

FIG. 5 is a conceptual diagram for explaining planes (also referred to as human body planes) set for a human body. In the present example embodiment, a sagittal plane dividing the body into the left half and the right half, a coronal plane dividing the body into the front half and the rear half, and a horizontal plane dividing the body horizontally are defined. As illustrated in FIG. 5, the world coordinate system and the local coordinate system coincide with each other in a state where the user stands upright with the center line of the foot facing the moving direction. In the present example embodiment, a rotation in the sagittal plane with the x axis as a rotation axis is defined as a roll, a rotation in the coronal plane with the y axis as a rotation axis is defined as a pitch, and a rotation in the horizontal plane with the z axis as a rotation axis is defined as a yaw. In addition, a rotation angle in the sagittal plane with the x axis as a rotation axis is defined as a roll angle, a rotation angle in the coronal plane with the y axis as a rotation axis is defined as a pitch angle, and a rotation angle in the horizontal plane with the z axis as a rotation axis is defined as a yaw angle.

As illustrated in FIG. 2, the feature amount data generation unit 12 (also referred to as a feature amount data generation device) includes an acquisition unit 121, a normalization unit 122, an extraction unit 123, a generation unit 125, and a feature amount data output unit 127. For example, the feature amount data generation unit 12 is achieved by a microcomputer or a microcontroller that performs overall control and data processing for the gait measurement device 10. For example, the feature amount data generation unit 12 includes a central processing unit (CPU), a random access memory (RAM), a read only memory (ROM), a flash memory, and the like. The feature amount data generation unit 12 controls the acceleration sensor 111 and the angular velocity sensor 112 to measure an angular velocity and an acceleration. For example, the feature amount data generation unit 12 may be implemented on a mobile terminal (not illustrated) carried by the subject (the user).

The acquisition unit 121 (acquisition means) acquires accelerations in three axial directions from the acceleration sensor 111. In addition, the acquisition unit 121 acquires angular velocities around three axes from the angular velocity sensor 112. For example, the acquisition unit 121 performs analog-to-digital conversion (AD conversion) on the acquired physical quantities (analog data) such as angular velocities and accelerations. Note that the physical quantities (analog data) measured by the acceleration sensor 111 and the angular velocity sensor 112 may be converted into digital data in the acceleration sensor 111 and the angular velocity sensor 112, respectively. The acquisition unit 121 outputs the converted digital data (also referred to as sensor data) to the normalization unit 122. The acquisition unit 121 may be configured to store the sensor data in a storage unit that is not illustrated. The sensor data includes at least acceleration data converted into digital data and angular velocity data converted into digital data. The acceleration data includes acceleration vectors in three axial directions. The angular velocity data includes angular velocity vectors around three axes. The acceleration data and the angular velocity data are associated with times at which the data are acquired. In addition, the acquisition unit 121 may apply a correction, such as a correction in mounting error or temperature or a correction in linearity, to the acceleration data and the angular velocity data.

The normalization unit 122 (normalization means) acquires the sensor data from the acquisition unit 121. The normalization unit 122 extracts time-series data (also referred to as gait waveform data) for one gait cycle from time-series data on accelerations in three axial directions and angular velocities around three axes included in the sensor data. The normalization unit 122 normalizes a time for the extracted gait waveform data for one gait cycle to a gait cycle of 0 to 100% (percent) (also referred to as first normalization). A timing such as 1% or 10% included in the gait cycle of 0 to 100% is also referred to as a gait phase. Furthermore, the normalization unit 122 normalizes the gait waveform data for one gait cycle subjected to the first normalization so that the stance phase is 60% and the swing phase is 40% (also referred to as second normalization). The stance phase is a period in which at least a partial portion of the back side of the foot is in contact with the ground. The swing phase is a period in which the back side of the foot is separated from the ground. By performing the second normalization on the gait waveform data, it is possible to suppress a difference of a gait phase in which a feature amount is extracted from being blurred due to the influence of disturbance.

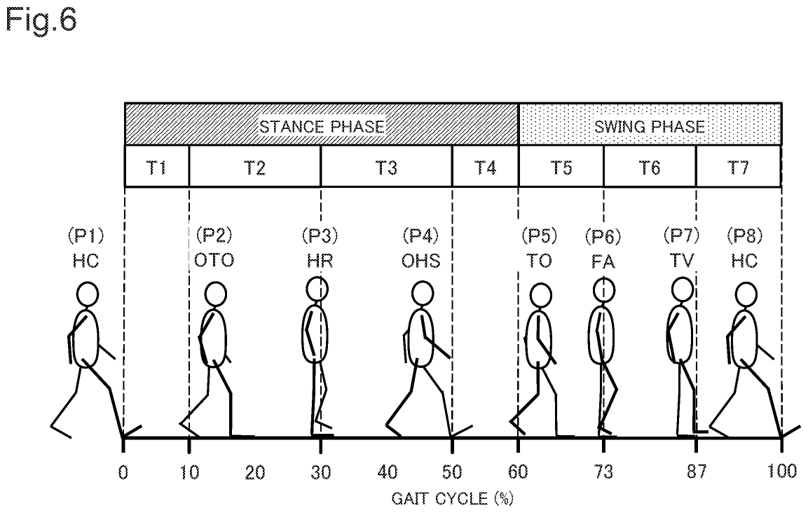

FIG. 6 is a conceptual diagram for explaining one gait cycle based on the right foot. One gait cycle based on the left foot is also similar to that based on the right foot. The horizontal axis of FIG. 6 represents one gait cycle of the right foot, with a time point at which the heel of the right foot lands on the ground as a start point and a next time point at which the heel of the right foot lands on the ground as an end point. In the horizontal axis in FIG. 6, first normalization is performed with one gait cycle as 100%. In addition, in the horizontal axis of FIG. 6, the second normalization is performed so that the stance phase is 60% and the swing phase is 40%. The one gait cycle of one foot is roughly divided into a stance phase, in which at least a partial portion of the back side of the foot is in contact with the ground, and a swing phase, in which the back side of the foot is separated from the ground. The stance phase is further subdivided into a load response period T1, a mid-stance period T2, a terminal stance period T3, and a pre-swing period T4. The swing phase is further subdivided into an initial swing period T5, a mid-swing period T6, and a terminal swing period T7. Note that FIG. 6 is an example, and does not limit the periods constituting one gait cycle, the names of these periods, and the like.

As illustrated in FIG. 6, in a gait, a plurality of events (also referred to as gait events) occur. P1 represents an event in which the heel of the right foot contacts the ground (heel contact (HC)). The heel contact HC is also expressed as heel strike HS. P2 represents an event in which the toe of the left foot is separated from the ground in a state where the sole of the right foot is in contact with the ground (opposite toe off (OTO)). P3 represents an event in which the heel of the right foot rises in a state where the sole of the right foot is in contact with the ground (heel rise (HR)). P4 is an event in which the heel of the left foot is in contact with the ground (opposite heel strike (OHS)). P5 represents an event in which the toe of the right foot is separated from the ground in a state where the sole of the left foot is in contact with the ground (toe off (TO)). P6 represents an event in which the left foot and the right foot cross each other in a state where the sole of the left foot is in contact with the ground (foot adjacent (FA)). P7 represents an event in which the tibia of the right foot is approximately perpendicular to the ground with the sole of the left foot in contact with the ground (tibia vertical (TV)). P8 represents an event in which the heel of the right foot is in contact with the ground (heel contact (HC)). P8 is an end point of the gait cycle starting from P1 and corresponds to a start point of a next gait cycle. Note that FIG. 6 is an example, and does not limit events that occur in a gait or the names of these events.

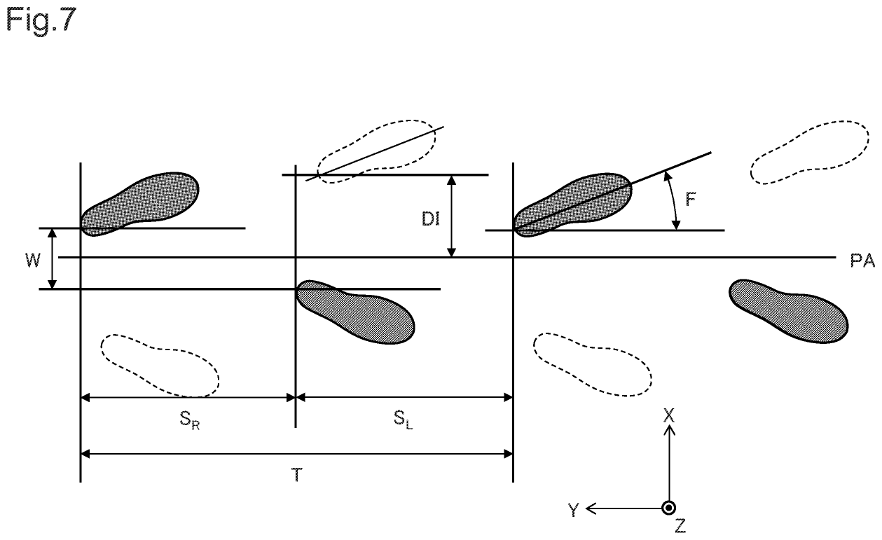

FIG. 7 is a conceptual diagram for explaining an example of a gait parameter. In FIG. 7, a right footstep length S_{R}, a left footstep length S_{L}, a stride length T, a step width W, a foot angle F, and a circumduction amount DI are illustrated. In addition, in FIG. 7, a movement axis PA is illustrated, the movement axis PA being parallel to the axis (Y axis) in the moving direction and corresponding to a trajectory continuing along a space between the left and right legs. The right footstep length S_{R} is a difference in the Y coordinate between the heel of the right foot and the heel of the left foot when the heel of the right foot swung out in the moving direction in a state where the sole of the left foot is in contact with the ground transitions to a landing state. The left footstep length S_{L} is a difference in the Y coordinate between the heel of the left foot and the heel of the right foot when the heel of the left foot swung out in the moving direction in a state where the sole of the right foot is in contact with the ground transitions to a landing state. The stride length Tis the sum of the right footstep length S_{R} and the left footstep length S_{L}. The step width W is an interval between the right foot and the left foot. In FIG. 7, the step width W is a difference between the center line (in the X coordinate) of the heel of the right foot in contact with the ground and the center line (in the X coordinate) of the heel of the left foot in contact with the ground. The foot angle F is an angle formed by the center line of the foot and the moving direction (the Y axis) in a state where the sole surface of the foot is in contact with the ground. In the present example embodiment, a foot angle in a state where the foot is in contact with the ground is evaluated in the stance phase. The circumduction amount DI is a distance between the movement axis PA and the foot at a timing when the central axis of the foot is farthest from the movement axis PA in the swing phase. In the present example embodiment, since the circumduction amount DI is affected by the length of the lower limb, the circumduction amount DI is normalized based on the height.

For example, the normalization unit 122 detects timings of heel contact HC and toe off TO from time-series data (solid line) on accelerations in the moving direction (accelerations in the Y direction). The timing of heel contact HC is a timing of a minimum peak immediately after a maximum peak appearing in the time-series data on accelerations in the moving direction (accelerations in the Y direction). The maximum peak serving as a mark of a timing of heel contact HC corresponds to a maximum peak of gait waveform data for one gait cycle. A section between consecutive heel contacts HC is one gait cycle. The timing of toe off TO is a rising timing of a maximum peak appearing after the period of the stance phase in which no fluctuations occur in time-series data on accelerations in the moving direction (accelerations in the Y direction).

For example, the normalization unit 122 detects a timing of a mid-stance period from time-series data (broken line) on roll angles (angular velocities around the X axis). A midpoint timing between a timing of a smallest roll angle and a timing of a largest roll angle corresponds to the mid-stance period. For example, parameters such as gait speed, stride, circumduction, internal/external rotation, and plantarflexion/dorsiflexion (also referred to as gait parameters) can be obtained based on the mid-stance period.

For example, the normalization unit 122 detects heel contact HC and toe off TO from the time-series data on accelerations in the moving direction (accelerations in the Y direction). The normalization unit 122 extracts a section between consecutive heel contacts HC as gait waveform data for one gait cycle. The normalization unit 122 converts the horizontal axis (time axis) of the gait waveform data for one gait cycle into a gait cycle of 0 to 100% by performing first normalization. The normalization unit 122 normalizes a section from the heel contact HC at which the gait phase is 0% to the toe off TO after the heel contact HC to 0 to 60%. In addition, the normalization unit 122 normalizes a section from the toe off TO to heel contact HC at which the gait phase is 100% after the toe off TO to 60 to 100%. As a result, the gait waveform data for one gait cycle is normalized to a section (a stance phase) in which the gait cycle is 0 to 60% and a section (a swing phase) in which the gait cycle is 60 to 100%. In the gait waveform data (solid line) after the second normalization, the timing of toe off TO coincides with 60%.

With respect to accelerations other than the accelerations in the moving direction (accelerations in the Y direction) and angular velocities, the normalization unit 122 extracts/normalizes gait waveform data for a gait cycle in line with the gait cycle for the accelerations in the moving direction (accelerations in the Y direction). Furthermore, the normalization unit 122 may generate time-series data on angles around three axes by integrating the time-series data on angular velocities around three axes. In this case, the normalization unit 122 also extracts/normalizes gait waveform data for one gait cycle, with respect to the angles around the three axes, in line with the gait cycle for the accelerations in the moving direction (accelerations in the Y direction).

The normalization unit 122 may extract/normalize gait waveform data for one gait cycle based on accelerations other than the accelerations in the moving direction (accelerations in the Y direction) and angular velocities. For example, the normalization unit 122 may detect heel contact HC and toe off TO from time-series data on accelerations in the vertical direction (accelerations in the Z direction). The timing of heel contact HC is a timing of a steep minimum peak appearing in the time-series data on accelerations in the vertical direction (accelerations in the Z direction). At the timing of the steep minimum peak, the value of the acceleration in the vertical direction (the acceleration in the Z direction) is substantially zero. The minimum peak serving as a mark of a timing of heel contact HC corresponds to a minimum peak of gait waveform data for one gait cycle. A section between consecutive heel contacts HC is one gait cycle. The timing of toe off TO is a timing of an inflection point in the middle in which fluctuations gradually increase after the time-series data on accelerations in the vertical direction (accelerations in the Z direction) passes through a section in which fluctuations are small after a maximum peak immediately after the heel contact HC. Furthermore, the normalization unit 122 may extract/normalize gait waveform data for one gait cycle based on both accelerations in the moving direction (accelerations in the Y direction) and accelerations in the vertical direction (accelerations in the Z direction). Furthermore, the normalization unit 122 may extract/normalize gait waveform data for one gait cycle based on accelerations other than the accelerations in the moving direction (the accelerations in the Y direction) and the accelerations in the vertical direction (the accelerations in the Z direction), angular velocities, angles, and the like.

The extraction unit 123 (extraction means) acquires the gait waveform data for one gait cycle normalized by the normalization unit 122. The extraction unit 123 extracts a feature amount to be used in estimating a falling risk factor (also referred to as a first feature amount) from the gait waveform data for one gait cycle. The feature amount extracted by the extraction unit 123 will be described in detail later. The extraction unit 123 extracts a feature amount for each gait phase cluster from gait phase clusters each being obtained by integrating temporally consecutive gait phases based on a preset condition. The gait phase cluster includes at least one gait phase. The gait phase cluster also includes a single gait phase. The gait waveform data or the gait phase as a source from which a feature amount to be used in estimating a falling risk factor is extracted will be described later.

The generation unit 125 (generation means) applies a feature amount constitutive formula to a feature amount (a first feature amount) extracted from each of the gait phases constituting the gait phase cluster to generate a feature amount (a second feature amount) of the gait phase cluster. The feature amount constitutive formula is a preset calculation formula for generating a feature amount of a gait phase cluster. For example, the feature amount constitutive formula is a calculation formula related to the four fundamental arithmetic operations. For example, the second feature amount calculated using the feature amount constitutive formula is an integral average value, an arithmetic average value, a slope, a variation, or the like between the first feature amounts of the respective gait phases included in the gait phase cluster. For example, the generation unit 125 applies a calculation formula for calculating a slope or a variation between the first feature amounts extracted from the respective the gait phases constituting the gait phase cluster as the feature amount constitutive formula. For example, in a case where the gait phase cluster is constituted by a single gait phase, it is not possible to calculate a slope or a variation, and thus, a feature amount constitutive formula for calculating an integral average value, an arithmetic average value, or the like may be used.

The feature amount data output unit 127 (feature amount data output means) outputs the feature amount data for each gait phase cluster generated by the generation unit 125 (also referred to as first feature amount data). The feature amount data output unit 127 outputs the generated feature amount data for the gait phase cluster to the estimation device 13 that executes estimation using the feature amount data.

### [Estimation Device]

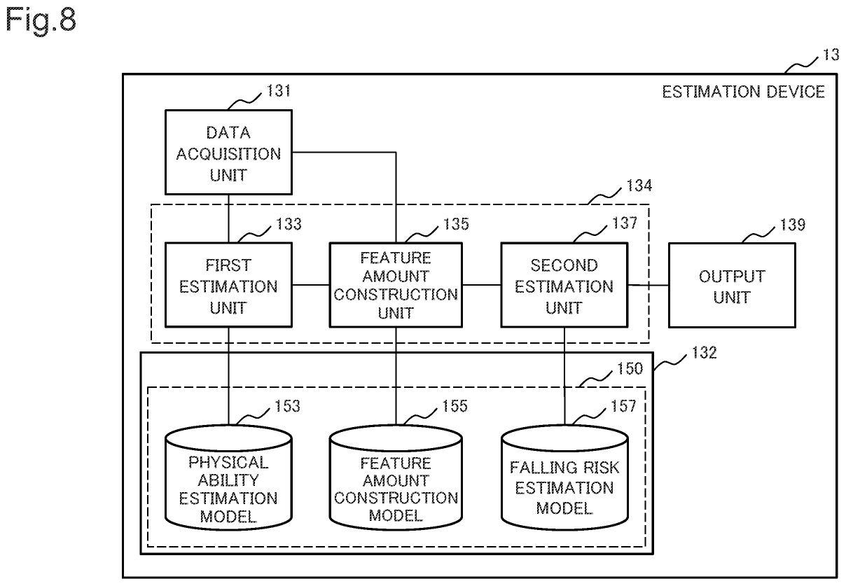

FIG. 8 is a block diagram illustrating an example of a configuration of the estimation device 13. The estimation device 13 includes a data acquisition unit 131, a storage unit 132, a first estimation unit 133, a feature amount construction unit 135, a second estimation unit 137, and an output unit 139. The first estimation unit 133, the feature amount construction unit 135, and the second estimation unit 137 constitute an estimation unit 134. In the present example embodiment, the estimation device 13 estimates a falling risk using the sensor data measured by the sensor 11 installed on the footwear worn by the subject. The estimation device 13 may be configured to estimate a falling risk using data measured by capturing a motion instead of the sensor data.

The data acquisition unit 131 (data acquisition means) acquires feature amount data calculated according to the gait of the subject from the gait measurement device 10. The feature amount data acquired by the data acquisition unit 131 is a vector including at least one feature amount to be used in estimating a physical ability. The data acquisition unit 131 outputs the feature amount data of the subject to the first estimation unit 133 and the feature amount construction unit 135.

In addition, the data acquisition unit 131 acquires attribute data of the subject. The attribute data includes a body mass index (BMI) and an age of the subject. The attribute data may include information on a gender or a physique of the subject. For example, the information on the physique includes a length of a leg, a thigh, a lower leg, a foot, or the like. For example, the attribute data is input via an input device (not illustrated). The attribute data may be stored in advance in the estimation device 13. The data acquisition unit 131 outputs the attribute data of the subject to the first estimation unit 133 and the feature amount construction unit 135.

For example, the data acquisition unit 131 receives the feature amount data from the gait measurement device 10 through wireless communication. The data acquisition unit 131 is configured to receive the feature amount data from the gait measurement device 10 through a wireless communication function (not illustrated) conforming to a standard such as Bluetooth (registered trademark) or Wi-Fi (registered trademark). Note that the communication function of the data acquisition unit 131 may conform to a standard other than Bluetooth (registered trademark) or Wi-Fi (registered trademark). The data acquisition unit 131 may be configured to receive the feature amount data from the gait measurement device 10 via a wire such as a cable.

The storage unit 132 (storage means) stores an estimation model 150. The estimation model 150 includes a physical ability estimation model 153, a feature amount construction model 155, and a falling risk estimation model 157. The storage unit 132 includes a plurality of storage areas. The physical ability estimation model 153, the feature amount construction model 155, and the falling risk estimation model 157 may be all stored in a single storage unit 132 or may be stored separately in a plurality of storage units 132. Each of the physical ability estimation model 153, the feature amount construction model 155, and the falling risk estimation model 157 will be described in detail later.

For example, each of the physical ability estimation model 153, the feature amount construction model 155, and the falling risk estimation model 157 is a model constructed in advance by machine learning to be described later with an input as an explanatory variable and each estimation target as a response variable. For example, these models are constructed by machine learning using a linear regression algorithm. For example, these models are constructed by machine learning using an algorithm of a support vector machine (SVM). For example, these models are constructed by machine learning using a Gaussian process regression (GPR) algorithm. For example, these models are constructed by machine learning using a random forest (RF) algorithm. For example, these models may be constructed, by unsupervised machine learning for classifying a subject who is a source from which the feature amount data is generated, according to the feature amount data. The machine learning algorithm for constructing these models is not particularly limited.

Each of the physical ability estimation model 153, the feature amount construction model 155, and the falling risk estimation model 157 may be stored in the storage unit 132 at the time of shipping a product from the factory, at the time of calibration before the user uses the estimation system 1, or the like. For example, each of the physical ability estimation model 153, the feature amount construction model 155, and the falling risk estimation model 157 may be a model stored in a storage device such as an external server. In that case, the estimation device 13 may be configured to use these models via an interface (not illustrated) connected to the storage device.

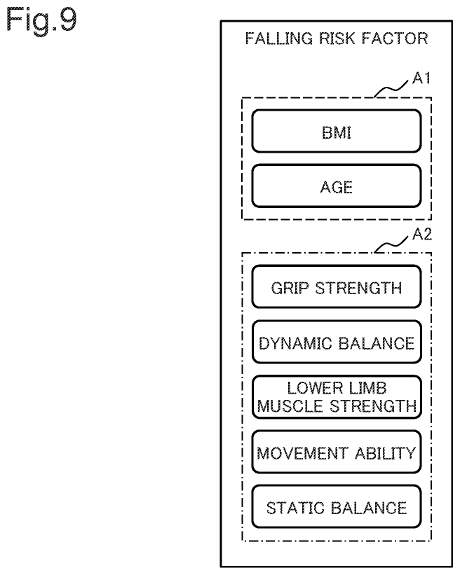

FIG. 9 is a conceptual diagram summarizing examples of falling risk factors. The falling risk factors include attribute factors A1 and physical ability factors A2. The attribute factors A1 are attribute data acquired by the data acquisition unit 131. In the example of FIG. 9, the attribute factors A1 include a BMI and an age. The physical ability factors A2 relate to physical abilities such as a grip strength, a dynamic balance, a lower limb muscle strength, a movement ability, and a static balance. These physical abilities correlate with a falling risk, and can be estimated based on the feature amount data measured by the gait measurement device 10.

The physical ability estimation model 153 estimates physical ability factors A2 using the feature amount data measured by the gait measurement device. The physical ability estimation model 153 is a model that estimates physical ability factors A2 learned with respect to a plurality of subjects. As the feature amount data is input, the physical ability estimation model 153 outputs an estimated value of each of the grip strength, the dynamic balance, the lower limb ability, the movement ability, and the static balance included in the physical ability factors A2. For example, the physical ability estimation model 153 is achieved by a single model. For example, the physical ability estimation model 153 may be configured by separate models for a plurality of physical abilities included in the physical ability factors A2, respectively. In that case, the models for the plurality of physical abilities may be distributed on separate pieces of hardware or integrated into a single piece of hardware.

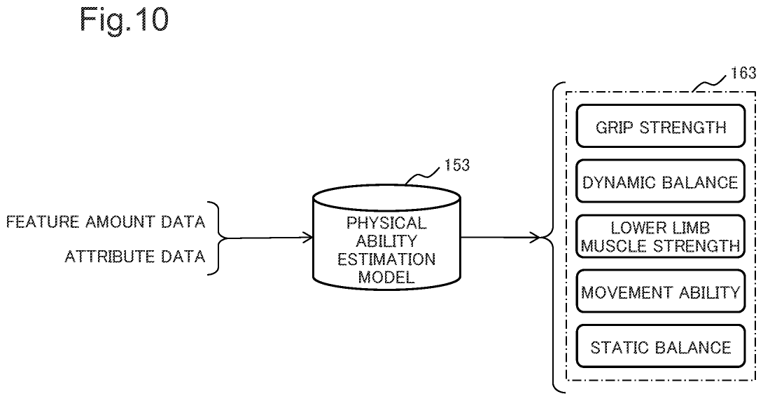

FIG. 10 is a conceptual diagram for explaining an example in which physical ability factors A2 are estimated using the feature amount data and the attribute data. In FIG. 10, the physical ability estimation model 153 used in estimating physical ability factors A2 are illustrated. In the example of FIG. 10, as the feature amount data and the attribute data are input, the physical ability estimation model 153 outputs estimated values 163 of physical ability factors A2. The estimated values 163 of the physical ability factors A2 include estimated values of the grip strength, the dynamic balance, the lower limb muscle strength, the movement ability, and the static balance. The estimated values 163 of the physical ability factors A2 are used for the feature amount construction unit 135 to construct feature amounts.

The grip strength, which is one of the physical ability factors A2, correlates with a total body muscle strength. The grip strength also correlates with a knee extension strength. In addition, the grip strength is affected by attributes such as gender, age, and height. In particular, the grip strength is affected by gender. For a man, there is a correlation between activities of quadriceps femoris muscles and the grip strength. Therefore, a feature related to the grip strength of the man is included in a gait phase in which a feature related to the activities of the quadriceps femoris muscles appears. For a woman, there is a correlation between activities of musculus vastus lateralis, musculus vastus intermedius, and musculus vastus medialis of the quadriceps femoris muscles and the grip strength. Therefore, a gait phase in which a feature related to the activities of musculus vastus lateralis, musculus vastus intermedius, and musculus vastus medialis appears includes a feature related to the grip strength of the woman.

Examples of the feature amounts related to the grip strength of the man include a feature amount AM1, a feature amount AM2, a feature amount AM3, and a feature amount AM4. The feature amount AM1 is extracted from a section for a gait phase of 3% of gait waveform data Ay related to time-series data on accelerations in the moving direction (accelerations in the Y direction). The gait phase of 3% is included in the load response period T1. The feature amount AM1 mainly includes a feature related to movements of musculus vastus lateralis, musculus vastus intermedius, and musculus vastus medialis among quadriceps femoris muscles. The feature amount AM2 is extracted from a section for gait phases of 59 to 62% of gait waveform data Ay related to time-series data on accelerations in the moving direction (accelerations in the Y direction). The gait phases of 59 to 62% are included in the pre-swing period T4. The feature amount AM2 mainly includes a feature related to a movement of musculus rectus femoris among quadriceps femoris muscles. The feature amount AM3 is extracted from a section for gait phases of 59 to 62% of gait waveform data Az related to time-series data on accelerations in the vertical direction (accelerations in the Z direction). The gait phases of 59 to 62% are included in the pre-swing period T4. The feature amount AM3 mainly includes a feature related to a movement of musculus rectus femoris among quadriceps femoris muscles. The feature amount AM4 is a ratio (DST1) of a period from the heel contact to the opposite toe off to a period in which both feet are simultaneously in contact with the ground (double support time (DST)). The DST1 is a ratio of the period from the heel contact to the opposite toe off to one gait cycle. The feature amount AM4 mainly includes a feature caused by quadriceps femoris muscles.

Examples of the feature amounts related to the grip strength of the woman include a feature amount AF1, a feature amount AF2, and a feature amount AF3. The feature amount AF1 is extracted from a section for a gait phase of 13% of gait waveform data Ax related to time-series data on accelerations in the lateral direction (accelerations in the X direction). The gait phase of 13% is included in the mid-stance period T2. The feature amount AF1 mainly includes a feature related to movements of musculus vastus lateralis, musculus vastus intermedius, and musculus vastus medialis among quadriceps femoris muscles. The feature amount AF2 is extracted from a section for gait phases 7 to 10% of gait waveform data Gy related to time-series data on angular velocities (pitch angular velocities) in the coronal plane (around the Y axis). The gait phases of 7 to 10% are included in the load response period T1. The feature amount AF2 mainly includes a feature related to movements of musculus vastus lateralis, musculus vastus intermedius, and musculus vastus medialis. The feature amount AF3 is a ratio (DST2) of a period from the opposite heel strike to the toe off to a period in which both feet are simultaneously in contact with the ground (double support time (DST)). The DST2 is a ratio of the period from the opposite heel strike to the toe off to one gait cycle. The sum of DST1 and DST2 corresponds to the period in both feet are simultaneously in contact with the ground in one gait cycle. The feature amount AF3 mainly includes a feature related to movements of musculus vastus lateralis, musculus vastus intermedius, and musculus vastus medialis.

As the feature amount AM1, the feature amount AM2, the feature amount AM3, and the feature amount AM4 are input, the physical ability estimation model 153 outputs an estimated value of the grip strength of the man. In addition, as the feature amount AF1, the feature amount AF2, and the feature amount AF3 acquired from the gait measurement device 10 are input, the physical ability estimation model 153 outputs an estimated value of the grip strength of the woman. The physical ability estimation model 153 may be configured to output a score according to a grip strength value instead of the estimated value of the grip strength. In this case, the score set according to the magnitude of the grip strength corresponds to the estimated value of the grip strength.

The dynamic balance, which is one of the physical ability factors A2, can be evaluated by a harmonic ratio (HR) of a waist. The harmonic ratios HR are calculated in three directions that are the left-right direction, the moving direction, and the vertical direction. The harmonic ratio HR in the moving direction corresponds to the smoothness of the movement of the waist in the front-back direction (in the sagittal plane) around the pelvis. The harmonic ratio HR in the left-right direction corresponds to the smoothness of the movement of the waist in the up-down direction (in the coronal plane) around the pelvis. The harmonic ratio HR in the vertical direction corresponds to the smoothness of the movement of the waist in the rotation (in the horizontal plane) of the body around the pelvis.

The harmonic ratio HR is an index focusing on the fact that one gait cycle is established by acceleration changes of two cycles including one cycle of one step of the right foot and one cycle of one step of the left foot. The harmonic ratio HR is an index related to a smoothness of a movement of a waist. A person with a small harmonic ratio HR may have a falling risk or have progressive low back pain. The left and right feet are connected to the pelvis through the lower legs and the thighs. The hip and knee joints are located between the left and right feet and the pelvis, but the periodicity of the pelvis and the periodicity of the waist in the gait is similar. Therefore, there is a phase in which the movement of the left and right feet and the movement of the waist are linked to each other. In the present example embodiment, the harmonic ratios related to the smoothness of the movement of the waist are estimated using sensor data measured according to a gait.

The harmonic ratios HR can be calculated using frequency components obtained by performing Fourier transform on time-series data on accelerations of the waist in one gait cycle. The frequency components include even-numbered (even Harmonics) frequency components (even components) corresponding to elements during the gait cycle and odd-numbered (odd Harmonics) frequency components (odd components) deviating from the even-numbered (even Harmonics) frequency components. The harmonic ratio HR is calculated for each of the vertical direction, the moving direction, and the left-right direction. Each of the harmonic ratios HR in the vertical direction and the moving direction is a ratio between the power sum of the even components and the power sum of the odd components. On the other hand, since one step of the left foot and one step of the right foot (two steps) in one gait cycle is one cycle, the harmonic ratio HR in the left-right direction is a ratio of the power sum of the odd components and the power sum of the even components. The left and right feet are connected to the pelvis through the lower legs and the thighs. The hip and knee joints are located between the left and right feet and the pelvis, but the periodicity of the pelvis and the periodicity of the waist in the gait is similar. Therefore, there is a phase in which the movement of the left and right feet and the movement of the waist are linked to each other.

As the frequency components of the sensor data (time-series data) measured by the gait measurement device 10 are input, the physical ability estimation model 153 outputs an estimated value of the dynamic balance. The physical ability estimation model 153 outputs estimated values of the harmonic ratios HR in the front-back direction, the left-right direction, and the vertical direction as the estimated value of the dynamic balance. The physical ability estimation model 153 may be configured to output odd components and even components of logarithmically transformed frequency components as feature amount data measured by the gait measurement device 10 is input. In that case, the first estimation unit 133 may be configured to calculate the harmonic ratios HR in the front-back direction, the left-right direction, and the vertical direction by using the odd components and the even components of the logarithmically transformed frequency components. The physical ability estimation model 153 may be configured to output a score according to the harmonic ratio HR instead of the harmonic ratio HR. In this case, the score set according to the value of the harmonic ratio HR corresponds to the estimated value of the dynamic balance.

The lower limb muscle strength, which is one of physical ability factors A2, can be evaluated by a grade of a chair stand-up test. For example, the chair stand-up test includes a five-time chair stand-up test in which standing up from and sitting down on a chair are repeated five times. The 5-time chair stand-up test is also referred to as a sit to stand-5 (SS-5) test. A grade of the five-time chair stand-up test is evaluated by a time for which standing up from and sitting down on a chair are repeated five times (also referred to as a standing-up and sitting-down time). The standing-up and sitting-down time is a grade value of the SS-5 test. The shorter the standing-up and sitting-down time, the higher the grade of the SS-5 test. The standing-up and sitting-down time may be evaluated by a grade of a 30-second chair stand-up (CS-30) test in which the number of times a motion of standing up from and sitting down on a chair is performed for 30 seconds is measured. The index of lower limb muscle strength is a standing-up and sitting-down time. The standing-up and sitting-down time correlates with quadriceps femoris muscles, hamstrings, anterior tibial muscles, and gastrocnemius muscles. Therefore, a feature related to the standing-up and sitting-down time is included in a gait phase in which these features appear.

Examples of the feature amounts related to the standing-up and sitting-down time include a feature amount C1, a feature amount C2, a feature amount C3, and a feature amount C4. The feature amount C1 is extracted from a section for gait phases of 42 to 54% of gait waveform data Gx related to time-series data on angular velocities in the sagittal plane (around the X axis). The gait phases of 42 to 54% are included in a section from the terminal stance period T3 to the pre-swing period T4. The feature amount C1 mainly includes a feature related to a movement of a gastrocnemius muscle. The feature amount C2 is extracted from a section for gait phases of 99 and 100% of gait waveform data Gy related to time-series data on angular velocities in the coronal plane (around the Y axis). The gait phases of 99 and 100% are included in the final stage of the terminal swing period T7. The feature amount C2 mainly includes a feature related to a movement of a quadriceps femoris muscle, a hamstring, or an anterior tibial muscle. The feature amount C3 is extracted from a section for gait phases of 10 to 12% of gait waveform data Gy related to time-series data on angular velocities in the coronal plane (around the Y axis). The gait phases of 10 to 12% are included in the early stage of the mid-stance period T2. The feature amount C3 mainly includes a feature related to a movement of a quadriceps femoris muscle, a hamstring, or a gastrocnemius muscle. The feature amount C4 is extracted from a section for a gait phase of 99% of gait waveform data Ez related to time-series data on angles (posture angles) in the horizontal plane (around the Z axis). The gait phase of 99% is included in the final stage of the terminal swing period T7. The feature amount C4 mainly includes a feature related to a movement of a quadriceps femoris muscle, a hamstring, or an anterior tibial muscle.

As the feature amount C1, the feature amount C2, the feature amount C3, and the feature amount C4 acquired from the gait measurement device 10 are input, the physical ability estimation model 153 outputs an estimated value of a grade value of the SS-5 test as an estimated value of the lower limb muscle strength. The physical ability estimation model 153 may be configured to output a score according to the grade value of the SS-5 test instead of the estimated value of the grade value of the SS-5 test. In this case, the score set according to the magnitude of the grade value of the SS-5 test corresponds to the estimated value of the lower limb muscle strength.

The movement ability, which is one of the physical ability factors A2, can be evaluated by a grade of a time up and go (TUG) test. For example, the grade of the TUG test can be evaluated by a time (also referred to as a TUG required time) required to stand up from a chair, walk to a mark 3 m (meter) ahead, change a direction, and sit down again on the chair. The TUG required time is a grade value of the TUG test. The shorter the TUG required time, the higher the grade of TUG test. The index of the movement ability is a TUG required time. The TUG required time correlates with quadriceps femoris muscles, gluteus medius muscles, and anterior tibial muscles. Therefore, a feature related to the TUG required time is included in a gait phase in which these features appear. A feature of the tensor fasciae latae muscle appears in gait phases of 0 to 45% and 85 to 100%. A feature of the gluteus medius muscle appears in gait phases of 0 to 25%. A feature of the anterior tibial muscle appears in gait phases 0 to 10% and 57 to 100%.

Examples of the feature amounts related to the movement ability include a feature amount D1, a feature amount D2, a feature amount D3, a feature amount D4, a feature amount D5, and a feature amount D6. The feature amount D1 is extracted from a section for gait phases of 64 and 65% of gait waveform data Ax related to time-series data on accelerations in the lateral direction (accelerations in the X direction). The gait phases of 64 and 65% are included in the initial swing period T5. The feature amount D1 mainly includes a feature related to a quadriceps femoris muscle in a standing-up and sitting-down motion. The feature amount D2 is extracted from a section for gait phases of 57 and 58% of gait waveform data Gx related to time-series data on angular velocities in the sagittal plane (around the X axis). The gait phases of 57 and 58% are included in the pre-swing period T4. The feature amount D2 mainly includes a feature related to a movement of a quadriceps femoris muscle associated with a foot kick-out speed. The feature amount D3 is extracted from a section for gait phases of 19 and 20% of gait waveform data Gy related to time-series data on angular velocities in the coronal plane (around the Y axis). The gait phases of 19 and 20% are included in the mid-stance period T2. The feature amount D3 mainly includes a feature related to a movement of a gluteus medius muscle during the change of the direction. The feature amount D4 is extracted from a section for gait phases of 12 and 13% of gait waveform data Ez related to time-series data on angular velocities in the horizontal plane (around the Z axis). The gait phases of 12 and 13% are included in the early stage of the mid-stance period T2. The feature amount D4 mainly includes a feature related to a movement of a gluteus medius muscle during the change of the direction. The feature amount D5 is extracted from a section for gait phases of 74 and 75% of gait waveform data Ez related to time-series data on angular velocities in the horizontal plane (around the Z axis). The gait phases of 74 and 75% are included in the early stage of the mid-swing period T6. The feature amount D5 mainly includes a feature related to a movement of an anterior tibial muscle during the standing-up and sitting-down and during the change of the direction. The feature amount D6 is extracted from a section for gait phases 76 to 80% of gait waveform data Ey related to time-series data on angles (posture angles) in the coronal plane (around the Y axis). The gait phases 76 to 80% are included in the mid-swing period T6. The feature amount D6 mainly includes a feature related to a movement of an anterior tibial muscle during the standing-up and sitting-down and during the change of the direction.

As the feature amount D1, the feature amount D2, the feature amount D3, the feature amount D4, the feature amount D5, and the feature amount D6 acquired from the gait measurement device 10 are input, the physical ability estimation model 153 outputs an estimated value of the TUG required time as an estimated value of the movement ability. The physical ability estimation model 153 may be configured to output a score according to the TUG required time instead of the estimated value of the TUG required time. In this case, the score set according to the length of the TUG required time corresponds to the estimated value of the movement ability.

The static balance, which is one of the physical ability factors A2, can be evaluated by a grade of a one-leg standing test. The grade of the one-leg standing test can be evaluated by a time (also referred to as a one-leg standing time) for which one leg is kept raised from the ground by 5 cm (centimeter) with the eyes being closed. The one-leg standing time is a grade value of the static balance. The larger the one-leg standing time, the higher the grade of the static balance. The static balance may be evaluated by a grade other than the grade of the eye-closed one-leg standing test. For example, the static balance may be evaluated by a one-leg standing test with eyes being opened (eye-opened one-leg standing test) or another modification of the one-leg standing test. The index of the static balance is a one-leg standing time. The one-leg standing time correlates with gluteus medius muscles, adductor longus muscles, sartorius muscles, and an inner-outer adductor muscle group. Therefore, a feature related to the one-leg standing time is included in a gait phase in which these features appear.

Examples of the feature amounts related to the static balance include a feature amount E1, a feature amount E2, a feature amount E3, a feature amount E4, a feature amount E5, a feature amount E6, and a feature amount E7. The feature amount E1 is extracted from a section for gait phases of 13 to 19% of gait waveform data Ax related to time-series data on accelerations in the lateral direction (accelerations in the X direction). The gait phases of 13 to 19% are included in the mid-stance period T2. The feature amount E1 mainly includes a feature related to a movement of a gluteus medius muscle. The feature amount E2 is extracted from a section for a gait phase of 95% of gait waveform data Az related to time-series data on accelerations in the vertical direction (accelerations in the Z direction). The gait phase 95% is included in the final stage of the terminal swing period T7. The feature amount E2 mainly includes a feature related to a movement of a gluteus medius muscle. The feature amount E3 is extracted from a section for gait phases of 64 and 65% of gait waveform data Gy related to time-series data on angular velocities in the coronal plane (around the Y axis). The gait phases of 64 and 65% are included in the initial swing period T5. The feature amount E3 mainly includes a feature related to movements of an adductor longus muscle and a sartorius muscle. The feature amount E4 is extracted from a section for gait phases of 11 to 16% of gait waveform data Gz related to time-series data on angular velocities in the horizontal plane (around the Z axis). The gait phases of 11 to 16% are included in the mid-stance period T2. The feature amount E4 mainly includes a feature related to a movement of a gluteus medius muscle. The feature amount E5 is extracted from a section for gait phases of 57 and 58% of gait waveform data Gz related to time-series data on angular velocities in the horizontal plane (around the Z axis). The gait phases of 57 and 58% are included in the pre-swing period T4. The feature amount E5 mainly includes a feature related to movements of an adductor longus muscle and a sartorius muscle. The feature amount E6 is extracted from a section for a gait phase of 100% of gait waveform data Ez related to time-series data on angles (posture angles) in the horizontal plane (around the Z axis). The gait phase of 100% corresponds to a timing of heel contact at which the terminal swing period T7 switches to the load response period T1. The feature amount of gait waveform data Ez in the gait phase of 100% corresponds to a foot angle in a state where the sole is in contact with the ground. The feature amount E6 mainly includes a feature related to a movement of a gluteus medius muscle. The feature amount E7 is a distance (circumduction amount) between the movement axis and the foot at a timing when the central axis of the foot is farthest from the movement axis in the swing phase. The feature amount E7 is a circumduction amount normalized by the height of the subject. The feature amount E7 mainly includes a feature related to a movement of an inner-outer adductor muscle group.

As the feature amount E1, the feature amount E2, the feature amount E3, the feature amount E4, the feature amount E5, the feature amount E6, and the feature amount E7 acquired from the gait measurement device 10 are input, the physical ability estimation model 153 outputs an estimated value of the static balance. The physical ability estimation model 153 outputs an estimated value of the one-leg standing time as an estimated value of the static balance. The physical ability estimation model 153 may be configured to output a score according to the one-leg standing time instead of the estimated value of the one-leg standing time. In this case, the score set according to the length of the one-leg standing time corresponds to the estimated value of the static balance.

The first estimation unit 133 (first estimation means) acquires feature amount data and attribute data from the data acquisition unit 131. The first estimation unit 133 estimates a physical ability factor A2 using the acquired feature amount data and attribute data. By inputting the feature amount data and the attribute data to the physical ability estimation model 153, the first estimation unit 133 estimates a physical ability factor A2 according to an output from the physical ability estimation model 153. The first estimation unit 133 outputs a result of estimating a physical ability factor A2 to the feature amount construction unit 135. In a case where a model stored in an external storage device constructed in a cloud, a server, or the like is used, the first estimation unit 133 is configured to use the model via an interface (not illustrated) connected to the storage device.

The feature amount construction model 155 is a model that constructs a feature amount (also referred to as a principal component) by performing principal component analysis on the estimated value of the physical ability factor A2 and the attribute data. For example, the feature amount construction model 155 is a principal component calculation formula constructed in advance based on machine learning data. As the estimated value of the physical ability factor A2 and the attribute data are input, the feature amount construction model 155 executes principal component analysis (PCA). The feature amount construction model 155 outputs a principal component vector (PCV) including at least one principal component. The feature amount construction model 155 may be configured to output only a principal component related to the falling risk factor.

For example, the feature amount construction model 155 constructs a principal component vector PSV by performing principal component analysis on indexes with respect to a plurality of subjects classified into two groups depending on whether they have experienced falling. For example, the feature amount construction model 155 outputs Cohen's d, which is an index for quantitatively evaluating a degree of separation of distribution between the two groups. Cohen's d is a standardized value (effect amount) obtained by dividing a difference in average value between two samples by a standard deviation. Cohen's d represents how much different the average values of the two samples are. The larger the value of Cohen's d, the larger the difference between the average values of the two samples, and thus, Cohen's d is effective in estimating a falling risk. The value output by the feature amount construction model 155 is not limited to a value of Cohen's d. For example, the feature amount construction model 155 may be configured to output g of Hedges.

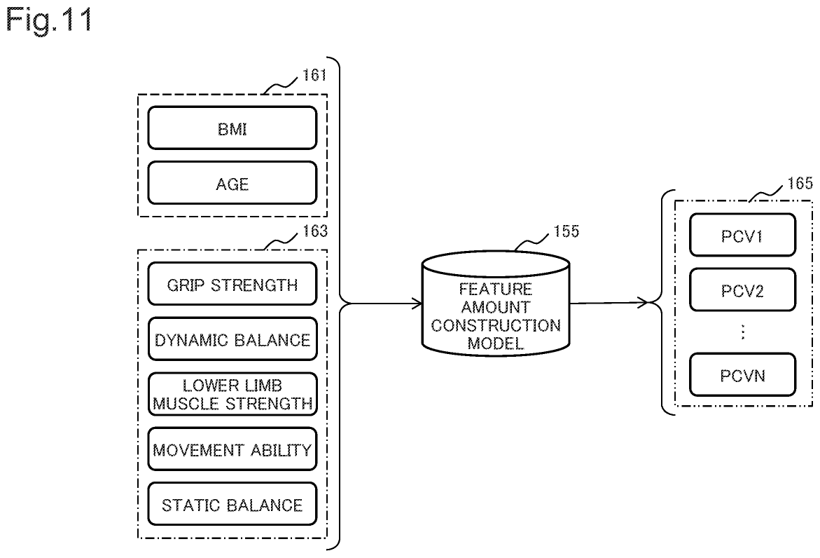

FIG. 11 is a conceptual diagram for explaining an example of principal component analysis PCA using the estimated value 163 of the physical ability factor A2 and the attribute data 161. In FIG. 11, the feature amount construction model 155 that executes principal component analysis PCA is illustrated. In the example of FIG. 11, as the estimated value 163 of the physical ability factor A2 and the attribute data 161 are input, the feature amount construction model 155 executes principal component analysis PCA and outputs at least one principal component (PCV_1, PCV_2, . . . , PCV_N) (Nis a natural number). As long as the falling risk factor can be discriminated, the number of principal components PCV output from the feature amount construction model 155 is not particularly limited. For example, the feature amount construction model 155 may be configured to output at least one principal component PCV as the attribute factor A1 and the score related to the physical ability factor A2 are input.

The feature amount construction unit 135 (feature amount construction means) constructs a feature amount to be used in estimating a falling risk factor using the estimated value 163 of the physical ability factor A2 and the attribute data 161. Specifically, the feature amount construction unit 135 inputs the estimated value 163 of the physical ability factor A2 and the attribute data 161 to the feature amount construction model 155, and constructs a principal component vector PSV output from the feature amount construction model 155 as feature amount data 165. The feature amount data 165 is a principal component vector PSV including at least one principal component. The feature amount construction unit 135 outputs the feature amount data 165 output from the feature amount construction model 155 to the second estimation unit 137. The feature amount construction unit 135 may be configured to output only a principal component to be used in estimating a falling risk factor, among the principal component vectors PSV included in the feature amount data 165 output from the feature amount construction model 155, to the second estimation unit 137.

The falling risk estimation model 157 estimates a score related to a falling risk factor (also referred to as a falling risk score) using the feature amount data 165 constructed by the feature amount construction unit 135. The falling risk estimation model 157 is a model that estimates a falling risk learned from a plurality of subjects. As the feature amount data 165 constructed by the feature amount construction unit 135 is input, the second estimation model outputs a falling risk score.

Furthermore, the falling risk estimation model 157 may be configured to output a countermeasure against the falling risk as the feature amount data 165 is input. The countermeasure against the falling risk estimated by the falling risk estimation model 157 is not particularly limited. For example, the falling risk estimation model 157 outputs a countermeasure for reducing the falling risk as the feature amount data 165 is input. For example, the falling risk estimation model 157 outputs a countermeasure for avoiding the falling risk as the feature amount data 165 is input. For example, the falling risk estimation model 157 outputs notification information for notifying a third party of the countermeasure for avoiding the falling risk as the feature amount data 165 is input.

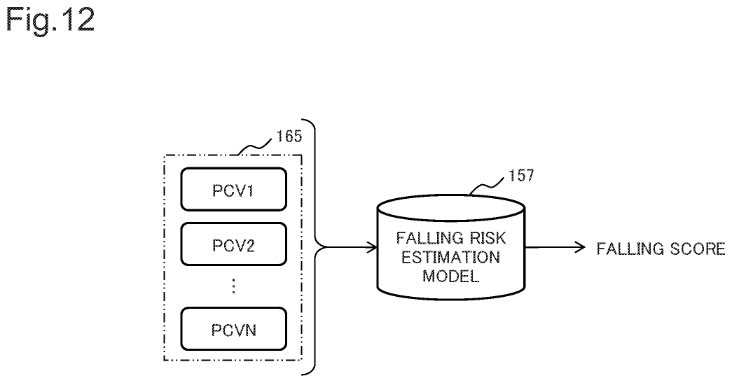

FIG. 12 is a conceptual diagram for explaining an example in which a falling score is estimated using the feature amount data 165 constructed by the feature amount construction unit 135. In the example illustrated in FIG. 12, the falling score is estimated using all of a plurality of feature amounts (principal components PCV) constructed by the feature amount construction unit 135. For example, the falling score may be estimated using some (principal components PCV) of the plurality of feature amounts (principal components PCV) constructed by the feature amount construction unit 135.

The second estimation unit 137 (second estimation means) acquires feature amount data (principal component PCV) from the feature amount construction unit 135. The second estimation unit 137 estimates a falling risk score using the acquired feature amount data (principal component PCV). By inputting the feature amount data (principal component PCV) to the falling risk estimation model 157, the second estimation unit 137 estimates the falling risk score output from the falling risk estimation model 157. The second estimation unit 137 outputs the estimated falling risk score to the output unit 139. In addition, the second estimation unit 137 acquires estimated values 163 of physical ability factors A2 from the feature amount construction unit 135. The second estimation unit 137 outputs any one of the acquired estimated values 163 of the physical ability factors A2 to the output unit 139.

The second estimation unit 137 may be configured to estimate a countermeasure against a falling risk based on the falling risk score and the estimated value 163 of the physical ability factor A2. In that case, the second estimation unit 137 does not particularly limit the countermeasure against the falling risk estimated by the second estimation unit 137. For example, when the falling risk score exceeds a predetermined threshold, the second estimation unit 137 outputs a countermeasure for reducing the falling risk and a countermeasure for avoiding falling. For example, when the falling risk score exceeds a predetermined threshold, the second estimation unit 137 outputs notification information for notifying a third party of the countermeasure for avoiding falling. In a case where the falling risk estimation model 157 is configured to output a countermeasure against a falling risk as the feature amount data 165 is input, the second estimation unit 137 is only required to output the countermeasure output from the falling risk estimation model 157.

The output unit 139 (output means) acquires falling risk information such as the falling risk score, the estimated value of the physical ability factor A2, and the countermeasure against the falling risk from the second estimation unit 137. The output unit 139 outputs the acquired falling risk information. For example, the output unit 139 displays the falling risk information on a screen of a mobile terminal of a subject (user). For example, the output unit 139 outputs the falling risk information to an external system or the like that uses the falling risk information. The use of the falling risk information output from the estimation device 13 is not particularly limited.

For example, the estimation device 13 is connected to an external system or the like constructed in a cloud or a server via a mobile terminal (not illustrated) carried by the subject (user). The mobile terminal (not illustrated) is a portable communication device. For example, the mobile terminal is a portable communication device having a communication function, such as a smartphone, a smart watch, or a mobile phone. For example, the estimation device 13 is connected to the mobile terminal via a wire such as a cable. For example, the estimation device 13 is connected to the mobile terminal through wireless communication. For example, the estimation device 13 is connected to the mobile terminal through a wireless communication function (not illustrated) conforming to a standard such as Bluetooth (registered trademark) or Wi-Fi (registered trademark). Note that the communication function of the estimation device 13 may conform to a standard other than Bluetooth (registered trademark) or Wi-Fi (registered trademark). A falling risk estimation result may be used by an application installed in the mobile terminal. In that case, the mobile terminal executes processing using the estimation result by application software or the like installed in the mobile terminal.

### [Principal Component Analysis]

Next, an example in which a principal component vector PSV is constructed by performing principal component analysis on the indexes with respect to a plurality of subjects classified into two groups depending on whether they have experienced falling will be described. Here, an example in which Cohen's d, which is an index for quantitatively evaluating a degree of separation of distribution between two groups, is used as an example of the principal component vector PSV will be described.

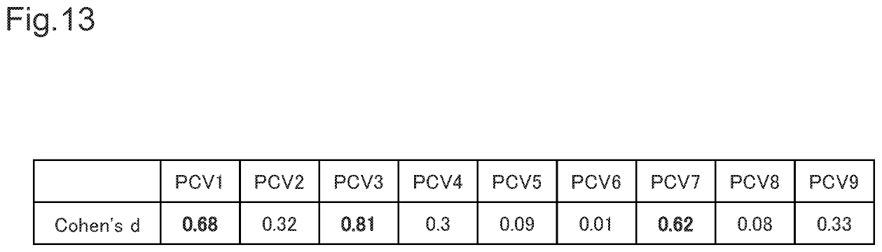

FIG. 13 is an example in which a principal component vector PSV constructed by performing principal component analysis on indexes with respect to a plurality of subjects classified into two groups depending on whether they have experienced falling. In the example illustrated in FIG. 13, the principal component analysis was performed with respect to 40 subjects having no experience of falling (non-falling group) and 24 subjects having experience of falling (falling group). In FIG. 13, Cohen's d values are calculated for nine principal components PCV1 to PCV9. The Cohen's d is calculated using the following Formula 1.

$$\begin{matrix}[ Formula 1 ] & \\ d = \frac{\vert {x}_{1} - {x}_{2} \vert}{{s}_{c}} & ( 1 )\end{matrix}$$

In the above Formula 1, x₁ represents a sample mean of a first group (non-falling group), and x₂ represents a sample mean of a second group (falling group). Sc is calculated using the following Formula 2.

$$\begin{matrix}[ Formula 2 ] & \\ {s}_{c} = \sqrt{\frac{{n}_{1} {s}_{22} + {n}_{2} {s}_{21}}{{n}_{1} + {n}_{2}}} & ( 2 )\end{matrix}$$

In the above Formula 2, n₁ represents a sample size of the first group (non-falling group), and S₂₁ represents a variance of the first group (non-falling group). In addition, n₂ represents a sample size, and S₂₂ represents a variance.

Regarding the Cohen's d in FIG. 13, values of PCV1, PCV3, and PCV7 are larger than those of the other PCVs. That is, Cohen's d values for PCV1, PCV3, and PCV7 are effective in estimating a falling risk.

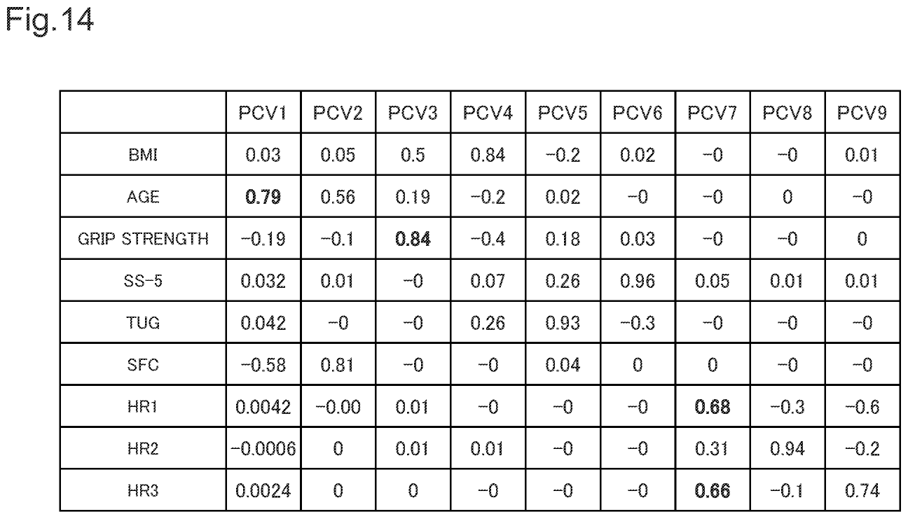

FIG. 14 is a table summarizing correlation coefficients between the principal components in FIG. 13 and the attribute factor A1 or the physical ability factor A2. The correlation coefficient is calculated in the middle of calculation for principal component analysis PCA, and is a value of a covariance matrix for reconstructing the score. The correlation coefficient indicates a correlation between the attribute factor A1 or the physical ability factor A2 as an input value and each PCV. The larger the absolute value of the correlation coefficient, the greater the correlation between the attribute factor A1 or the physical ability factor A2 and each PCV. In the table of FIG. 14, the harmonic ratio HR, which is an index of the dynamic balance, is divided into three directions: a harmonic ratio HR1 in the vertical direction, a harmonic ratio HR2 in the left-right direction, and a harmonic ratio HR3 in the moving direction.

According to the table of FIG. 14, the correlation coefficients for PCV1, PCV3, and PCV7 show high values of 0.6 or more. PCV1 has a high correlation with the age, and thus is called an aging factor. PCV3 has a high correlation with the grip strength, and thus is referred to as a grip strength factor. PCV7 has a high correlation with the harmonic ratio HR (dynamic balance), and thus is called a dynamic balance factor.

Next, based on the table of FIG. 14, an example in which the generalization performance of the falling risk estimation model 157 trained by using PCV1, PCV3, and PCV7 alone or in combination was verified by k-split cross validation is illustrated.

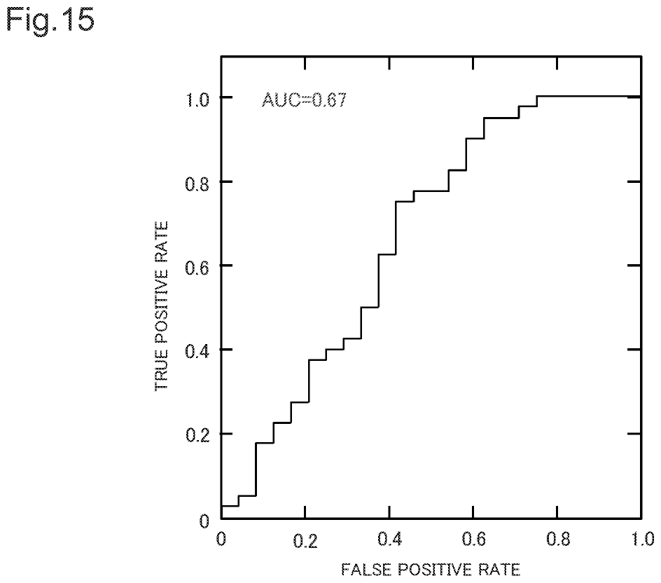

FIGS. 15 to 17 are receiver operating characteristic (ROC) curves each showing a result of 10-split cross validation performed using 64 training data sets for the 40 subjects in the non-falling group and the 24 subjects in the falling group described above. In the graphs of FIGS. 15 to 17, the horizontal axis represents a false positive rate, and the vertical axis represents a true positive rate. The larger the area under the ROC curve (area under the curve (AUC)), the higher the accuracy of the falling risk estimation model 157.

FIG. 15 is an ROC curve showing a result of performing 10-split cross validation on a falling risk estimation model 157 constructed by machine learning using a grip strength factor (PCV3). The example of verification in FIG. 15 is an example of the falling risk estimation model 157 constructed by machine learning using, as a second feature amount, one principal component of which an index indicating a degree of separation of distribution between the two groups into which the subjects are classified depending on whether they have experienced falling exceeds a predetermined value. As a result of 10-split cross validation on the falling risk estimation model 157 constructed by machine learning using only the grip strength factor (PCV3), the AUC was 0.67.

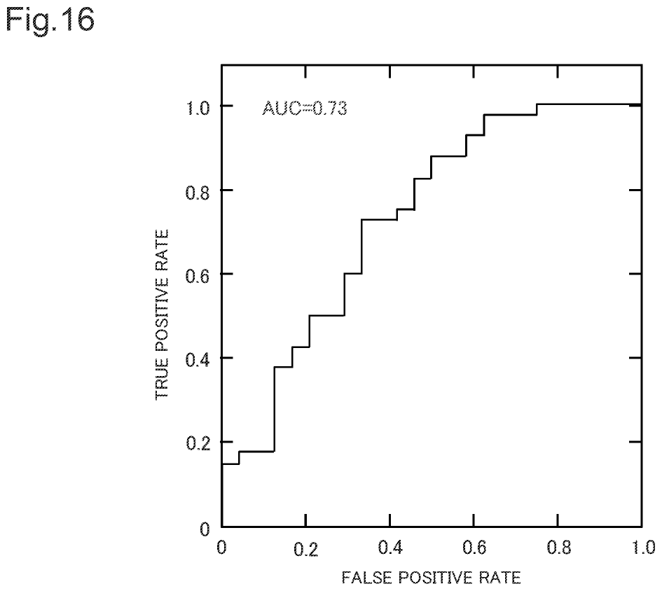

FIG. 16 is an ROC curve showing a result of performing 10-split cross validation on a falling risk estimation model 157 constructed by machine learning using an aging factor (PCV1) and a grip strength factor (PCV3). The example of verification in FIG. 16 is an example of the falling risk estimation model 157 constructed by machine learning using, as a second feature amount, two principal components each of which an index indicating a degree of separation of distribution between the two groups into which the subjects are classified depending on whether they have experienced falling exceeds a predetermined value. As a result of 10-split cross validation on the falling risk estimation model 157 constructed by machine learning using the aging factor (PCV1) and the grip strength factor (PCV3), the AUC was 0.73.

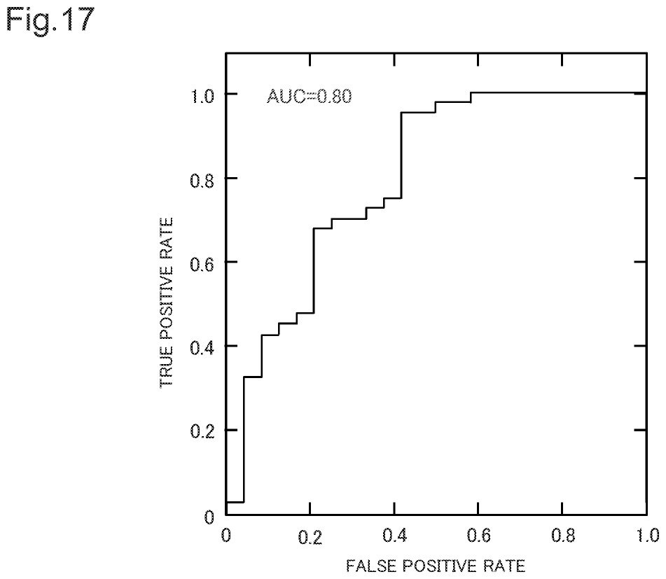

FIG. 17 is an ROC curve showing a result of performing 10-split cross validation on a falling risk estimation model 157 constructed by machine learning using an aging factor (PCV1), a grip strength factor (PCV3), and a dynamic balance factor (PCV7). The example of verification in FIG. 17 is an example of the falling risk estimation model 157 constructed by machine learning using, as a second feature amount, three principal components each of which an index indicating a degree of separation of distribution between the two groups into which the subjects are classified depending on whether they have experienced falling exceeds a predetermined value. As a result of 10-split cross validation on the falling risk estimation model 157 constructed by machine learning using the aging factor (PCV1), the grip strength factor (PCV3), and the dynamic balance factor (PCV7), the AUC was 0.80.

As a result of the verification in FIGS. 15 to 17, the accuracy of the falling risk estimation model 157 constructed by combining a plurality of physical ability factors was higher than that of the falling risk estimation model 157 constructed using a single physical ability factor. That is, the accuracy of the falling risk estimation model 157 is higher when the falling risk estimation model is constructed by combining a plurality of physical ability factors.

### [Falling Risk]

Next, an example in which a falling risk score is calculated for the above-described subject group (64 subjects) including the non-falling group (40 subjects) and the falling group (24 subjects) will be described. Here, in the example, a falling risk is expressed by a posterior probability calculated using Fisher's linear discriminant analysis and Mahalanobis distance. In the example, the following calculations are performed using commercially available numerical analysis software (Matlab: registered trademark). In the following example, the non-falling group is defined as class 1, and the falling group is defined as class 2.

The prior probabilities Prior of class 1 (non-falling group) and class 2 (falling group) in the subject group (64 subjects) are expressed by the following Formula 3.

$$\begin{matrix}[ Formula 3 ] & \\ Prior = [ 0.625 \text{.0} \text{.375} ] & ( 3 )\end{matrix}$$

A logarithm LogDetSigma of a determinant for an inter-class covariance matrix of class 1 (non-falling group) and class 2 (falling group) is 4.429.

A class average Mu1 of input values (PCV1, PCV3, PCV7) for class 1 (non-falling group) is expressed by the following Formula 4.

$$\begin{matrix}[ Formula 4 ] & \\ Mu = [ - 2.7226 , 1.2476 , 0.0461 ] & ( 4 )\end{matrix}$$

A class average Mu2 of input value (PCV1, PCV3, PCV7) for class 2 (falling group) is expressed by the following Formula 5.

$$\begin{matrix}[ Formula 5 ] & \\ Mu = [ 4.5377 , - 2.0785 , - 0.0768 ] & ( 5 )\end{matrix}$$

The covariance matrix R of class 1 (non-falling group) and class 2 (falling group) is expressed by the following Formula 6.

$$\begin{matrix}[ Formula 6 ] & \\ InvR = [ \begin{matrix}- 0.5056 & 0.5874 & 06579 \\ - 0.7377 & - 0.1215 & - 0.6924 \\ 0.2801 & 0.7401 & - 0.6359\end{matrix} ] & ( 6 )\end{matrix}$$

The Mahalanobis distance Mah1 of class 1 (non-falling group) is expressed by the following Formula 7.

$$\begin{matrix}[ Formula 7 ] & \\ Mah 1 = {sum ( ( Score 1 - Mu 1 ) \times InvK )}^{2} & ( 7 )\end{matrix}$$

The Mahalanobis distance Mah2 of class 2 (non-falling group) is expressed by the following Formula 8.

$$\begin{matrix}[ Formula 8 ] & \\ Mah 2 = {sum ( ( Score 2 - Mu 2 ) \times InvK )}^{2} & ( 8 )\end{matrix}$$

In the above Formulas 7 and 8, InvK is a square root of an inverse matrix for the covariance of each of class 1 (non-falling group) and class 2 (falling group).

Here, the variable P is defined for each class by the following Formula 9.

$$\begin{matrix}[ Formula 9 ] & \\ log P = log ( Prior ) - \frac{Log DetSigma}{2} - \frac{[ Mah 1 , Mah 2 ]}{2} & ( 9 )\end{matrix}$$

The logP is a vector of one row and two columns.

The larger one of the two elements is extracted by the calculation using the following Formula 10.

$$\begin{matrix}[ Formula 10 ] & \\ max Log P = max ( log P ) & ( 10 )\end{matrix}$$

By transforming the above Formula 10, the following Formula 11 is obtained.

$$\begin{matrix}[ Formula 11 ] & \\ P = exp ( log P - max Log P ) & ( 11 )\end{matrix}$$

The posterior probabilities Posterior of class 1 (non-falling group) and class 2 (falling group) are calculated using the following Formula 12.

$$\begin{matrix}[ Formula 12 ] & \\ Posterior = P / sum ( P ) & ( 12 )\end{matrix}$$

In the above Formula 12, sum(P) is the sum of all elements of P.

The posterior probability Posterior(2) of class 2 (falling group) is a falling risk score.

Next, a result of comparison between a model constructed using grip strengths (true values) measured in actual physical ability tests and a model constructed by the method according to the present example embodiment will be described.

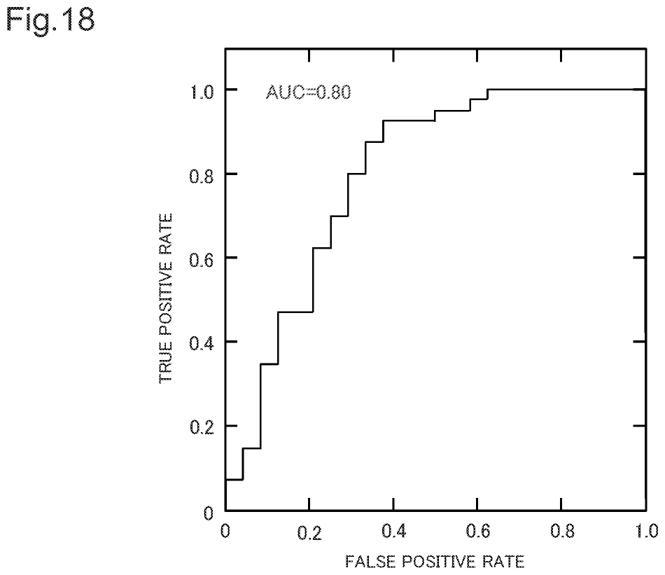

FIG. 18 is an ROC curve showing a result of performing 10-split cross validation on the falling risk estimation model 157 trained using the grip strengths (true values) measured in the actual physical ability tests. In FIG. 18, the horizontal axis represents a false positive rate, and the vertical axis represents a true positive rate. As a result of 10-split cross validation on the falling risk estimation model 157 constructed by machine learning using the grip strengths (true values), the AUC was 0.80.

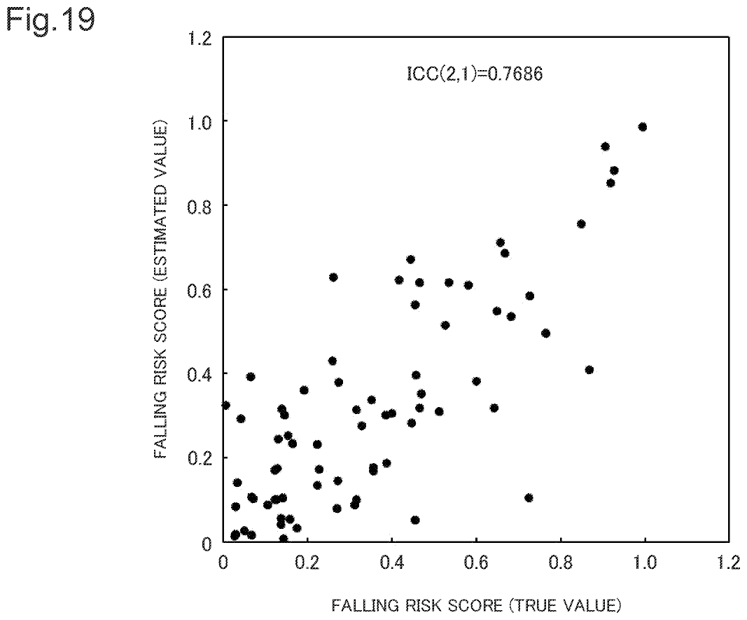

FIG. 19 is a graph showing a correlation between a falling risk score (true value) estimated by a model constructed using actually measured values (true values) of grip strengths and a falling risk score (estimated value) estimated by a method according to the present example embodiment: In FIG. 19, the horizontal axis represents a score (true value), and the vertical axis represents a score (estimated value). An intraclass correlation coefficient ICC(2,1) between the falling risk score (true value) and the falling risk score (estimated value) was 0.7686. That is, there is a sufficient correlation between the falling risk score (true value) and the falling risk score (estimated value).

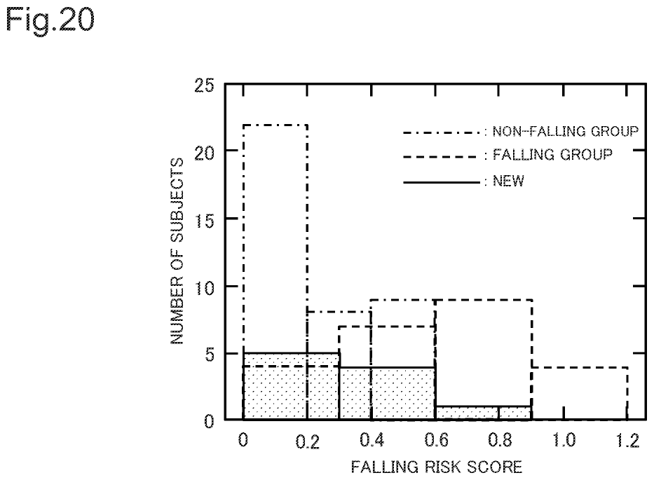

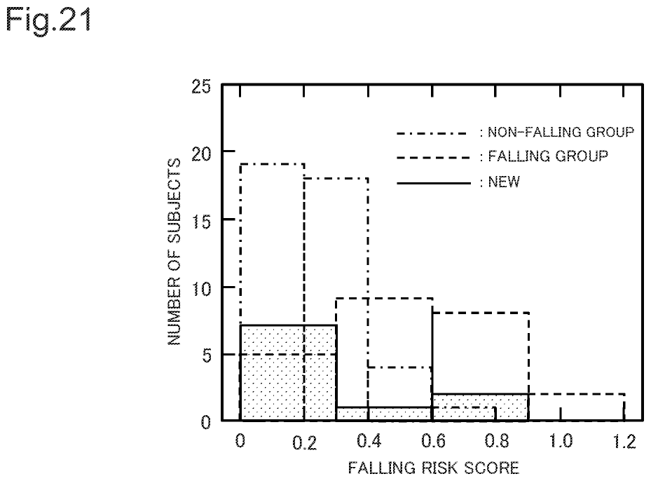

FIGS. 20 to 21 are graphs showing frequency distributions of falling risk scores estimated by the falling risk estimation model 157 constructed using grip strengths (true values) and grip strengths (estimated values), including new subjects. A dashed line is a frequency distribution of falling risk scores for the subjects (24 subjects) in the falling group. A dashed-dotted line is a frequency distribution of falling risk scores for the subjects (40 subjects) in the non-falling group. A solid line is a frequency distribution of falling risk scores for new subjects (10 subjects).

FIG. 20 is a graph showing a frequency distribution of falling risk scores estimated by the falling risk estimation model 157 constructed using grip strengths (true values). FIG. 21 is a graph showing a frequency distribution of falling risk scores estimated by the falling risk estimation model 157 constructed using grip strengths (estimated values). Upon comparing FIG. 20 and FIG. 21, a similar distribution is obtained for a falling risk estimation model 157 constructed using either grip strengths (true values) or grip strengths (estimated values). In addition, since a similar distribution is obtained for new subjects not used for machine learning, the falling risk estimation model 157 constructed using the method according to the present example embodiment has a sufficient generalization performance.

### (Operation)

Next, an operation of the estimation system 1 will be described with reference to the drawings. Hereinafter, the gait measurement device 10 and the estimation device 13 included in the estimation system 1 will be individually described. Concerning the gait measurement device 10, an operation of the feature amount data generation unit 12 included in the gait measurement device 10 will be described.

### [Gait Measurement Device]

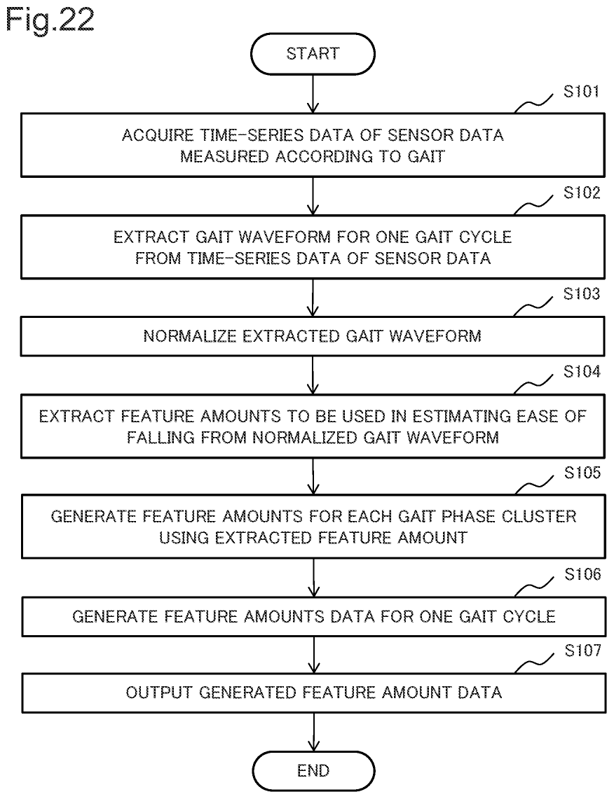

FIG. 22 is a flowchart for explaining an example of the operation of the gait measurement device 10. In the description based on the flowchart of FIG. 22, the feature amount data generation unit 12 included in the gait measurement device 10 will be described as an operation subject.

In FIG. 22, first, the feature amount data generation unit 12 acquires time-series data of sensor data measured according to a gait (step S101).

Next, the feature amount data generation unit 12 extracts gait waveform data for one gait cycle from the time-series data of the sensor data (step S102). The feature amount data generation unit 12 detects heel contact and toe off from the time-series data of the sensor data. The feature amount data generation unit 12 extracts time-series data for a section between consecutive heel contacts as the gait waveform data for one gait cycle.

Next, the feature amount data generation unit 12 normalizes the extracted gait waveform data for one gait cycle (step S103). The feature amount data generation unit 12 normalizes the gait waveform data for one gait cycle to a gait cycle of 0 to 100% (first normalization). Furthermore, the feature amount data generation unit 12 normalizes a ratio of a stance phase to a swing phase with respect to the gait waveform data for one gait cycle subjected to the first normalization to 60:40 (second normalization).

Next, the feature amount data generation unit 12 extracts feature amounts from gait phases to be used in estimating a falling risk with respect to the normalized gait waveform (step S104). For example, the feature amount data generation unit 12 extracts feature amounts input to an estimation model (first estimation model) constructed in advance.

Next, the feature amount data generation unit 12 generates feature amounts for each gait phase cluster using the extracted feature amounts (step S105).

Next, the feature amount data generation unit 12 generates feature amount data for one gait cycle by integrating the feature amounts for each gait phase cluster (step S106).

Next, the feature amount data generation unit 12 outputs the generated feature amount data to the estimation device 13 (step S107).

### [Estimation Device]

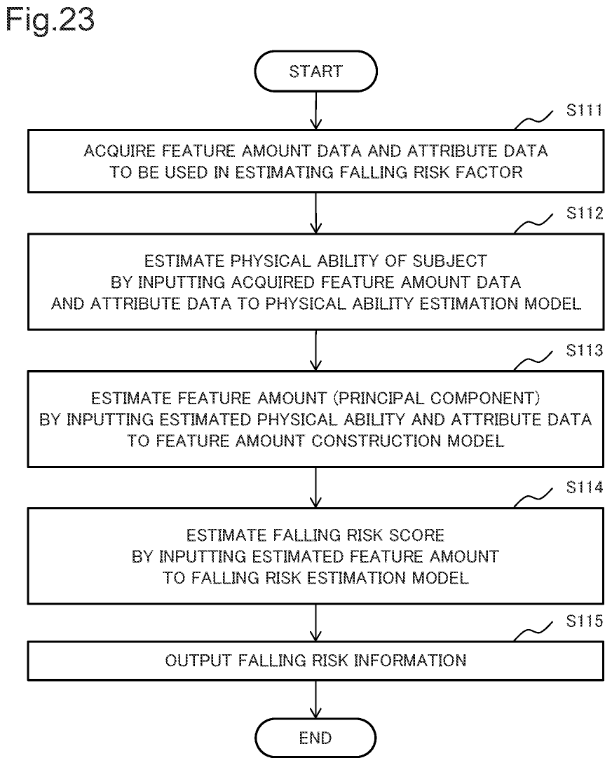

FIG. 23 is a flowchart for explaining an example of the operation of the estimation device 13. In the description based on the flowchart of FIG. 23, the estimation device 13 will be described as an operation subject.

In FIG. 23, first, the estimation device 13 acquires feature amount data generated using sensor data measured according to gait (step S111).

Next, the estimation device 13 estimate a physical ability of a subject by inputting the acquired feature amount data and attribute data to the physical ability estimation model 153 (step S112).

Next, the estimation device 13 estimates a feature amount (principal component) by inputting the estimated physical ability and the attribute data to the feature amount construction model 155 (step S113).

Next, the estimation device 13 estimates a falling risk score by inputting the estimated feature amount to the falling risk estimation model 157 (step S114).

Next, the estimation device 13 outputs falling risk information such as the estimated falling risk score, an estimated value of a physical ability factor A2, and a countermeasure against a falling risk (step S115). For example, the falling risk information is output to a terminal device (not illustrated) carried by the user. For example, the falling risk information is output to a system that executes processing using the falling risk information.

### (Example of Application)

Next, an example of application according to the present example embodiment will be described with reference to the drawings. In the following example of application, an example in which the estimation device 13 installed in the mobile terminal carried by the user estimates falling risk information using feature amount data measured by the gait measurement devices 10 arranged in shoes will be described.

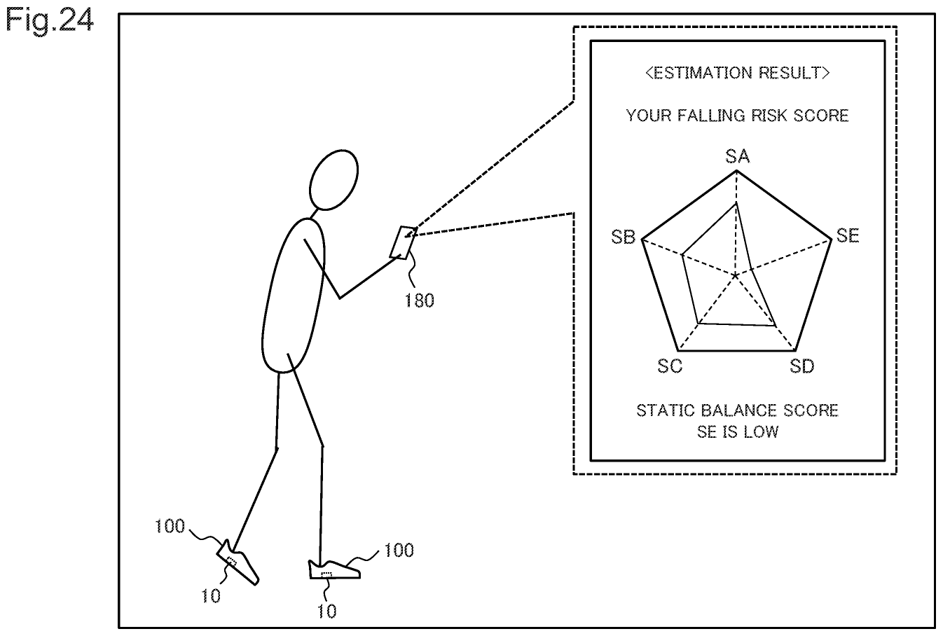

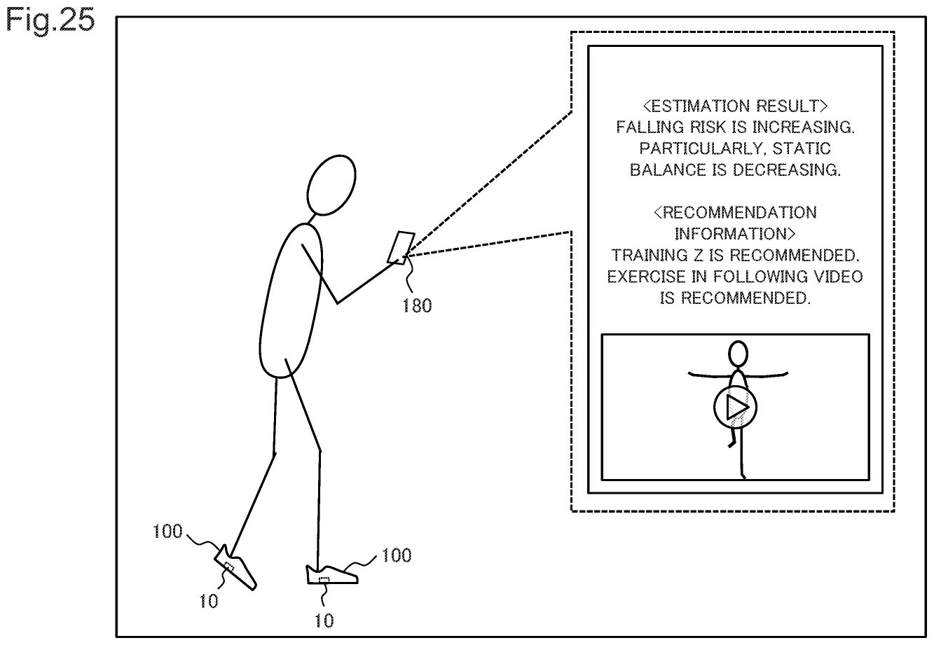

FIGS. 24 and 25 are conceptual diagrams each illustrating an example in which a result of estimation by the estimation device 13 is displayed on a screen of a mobile terminal 180 carried by the user who walks while wearing shoes 100 in which the gait measurement devices 10 are arranged. In the example of each of FIGS. 24 and 25, falling risk information according to a result of estimating a falling risk using feature amount data based on sensor data measured according to a gait of the user is displayed on the screen of the mobile terminal 180.

FIG. 24 is an example in which falling risk information is displayed on the screen of the mobile terminal 180. In the example of FIG. 24, a radar chart according to a value of a score for each physical ability factor A2 is displayed on the screen of the mobile terminal 180. SA is a score related to grip strength (total muscle strength). SB is a score related to dynamic balance. SC is a score related to lower limb muscle strength. SD is a score related to movement ability. SE is a score related to static balance. In the example of FIG. 24, physical ability-related information, “The static balance score SE is low.”, is displayed on the screen of the mobile terminal 180. The user who has seen the falling risk information displayed on the display unit of the mobile terminal 180 can see that the static balance score SE is lower as compared to the scores of the other physical ability factors A2.

The estimated falling risk information may be provided to one other than the user. For example, the falling risk information may be output to a trainer that manages a physical condition of the user or a terminal device (not illustrated) used by a family member of the user or the like. For example, the falling risk information may be recorded in a database (not illustrated) constructed for the purpose of health management or the like.

FIG. 25 illustrates another example in which falling risk information according to a falling risk estimation result is displayed on the screen of the mobile terminal 180. In the example of FIG. 25, falling risk information, “The falling risk is increasing.”, is displayed on the screen of the mobile terminal 180 according to a falling risk estimation result. Furthermore, in the example of FIG. 25, information regarding the static balance of which a score was low, among the five physical ability factors A2 used in the estimation of the falling risk, is displayed on the display unit of the mobile terminal 180. In the example of FIG. 25, information regarding the static balance, “The static balance is decreasing”, is displayed on the display unit of the mobile terminal 180 according to the estimated value of the static balance score SE. Furthermore, in the example of FIG. 25, according to the estimated value of the static balance score SE, recommendation information according to the static balance estimation result, “Training Z is recommended. Please see the video below.”, is displayed on the display unit of the mobile terminal 180. The user who has seen the information displayed on the display unit of the mobile terminal 180 can practice training leading to an increase in static balance by exercising with reference to the video for the training Z according to the recommendation information.

As described above, the estimation system according to the present example embodiment includes a gait measurement device and an estimation device. The gait measurement device is installed on footwear of a subject who is a target in estimating a falling risk factor. The gait measurement device includes a sensor and a feature amount data generation unit. The sensor includes an acceleration sensor and an angular velocity sensor. The sensor measures a spatial acceleration and a spatial angular velocity. The sensor generates sensor data according to a gait using the measured spatial acceleration and spatial angular velocity, and outputs the generated sensor data. The feature amount data generation unit extracts gait waveform data for one gait cycle from time-series data of the sensor data. The feature amount data generation unit normalizes the extracted gait waveform data, and extracts a first feature amount to be used in estimating a falling risk factor from the normalized gait waveform data. The feature amount data generation unit generates first feature amount data including the extracted first feature amount, and outputs the generated first feature amount data to the estimation device.

The estimation device includes a data acquisition unit, a storage unit, an estimation unit, and an output unit. The data acquisition unit acquires first feature amount data related to a physical ability measured according to a gait of the subject and attribute data of the subject. The storage unit stores an estimation model. The estimation model estimates at least one physical ability factor related to the falling risk factor as the first feature amount data and the attribute data are input. The estimation model constructs a second feature amount by performing principal component analysis on an attribute factor included in the attribute data and the estimated physical ability factor. The estimation model outputs a falling risk score using the constructed second feature amount. The estimation unit inputs the acquired first feature amount data and the acquired attribute data to the estimation model, and estimates falling risk information using at least one physical ability factor output from the estimation model. The output unit outputs the estimated falling risk information.

The estimation system according to the present example embodiment generates first feature amount data related to a physical ability of a subject through a gait measurement device installed on footwear of the subject. The estimation system according to the present example embodiment estimates a falling risk for the subject using the generated first feature amount data and attribute data of the subject. That is, according to the present example embodiment, the falling risk can be estimated using the physical ability-related data measured according to the gait.

In an aspect of the present example embodiment, the data acquisition unit acquires first feature amount data. The first feature amount data is used to estimate at least one physical ability factor related to a falling risk factor, the physical ability factor being extracted from gait waveform data generated using time-series data of sensor data measured according to a gait. The storage unit stores a physical ability estimation model, a feature amount construction model, and a falling risk estimation model. The physical ability estimation model outputs at least one physical ability factor as the first feature amount data and attribute data are input. As at least one attribute factor and at least one physical ability factor are input, the feature amount construction model performs principal component analysis on the at least one attribute factor and the at least one physical ability factor, and outputs at least one second feature amount. The falling risk estimation model outputs a falling risk score as at least one second feature amount is input. The estimation unit includes a first estimation unit, a feature amount construction unit, and a second estimation unit. The first estimation unit inputs the first feature amount data and the attribute data to the physical ability estimation model, and estimates at least one physical ability factor output from the physical ability estimation model as a physical ability of the subject. The feature amount construction unit constructs at least one second feature amount by inputting at least one attribute factor and at least one physical ability factor to the feature amount construction model.

The second estimation unit inputs at least one second feature amount to the falling risk estimation model, and estimates falling risk information related to the subject using a falling risk score output from the falling risk estimation model.

According to the present aspect, the falling risk information including the physical ability factor and the falling risk score can be estimated using the physical ability estimation model, the feature amount construction model, and the falling risk estimation model.

In an aspect of the present example embodiment, the attribute data includes a body mass index (BMI) and an age of the subject as the attribute factor. According to the present aspect, the falling risk information can be estimated using data including the attribute factors of the subject.

In an aspect of the present example embodiment, the physical ability estimation model outputs, as the physical ability factor, estimated values related to a grip strength, a dynamic balance, a lower limb muscle strength, a movement ability, and a static balance as the attribute data and the first feature amount data are input. Therefore, according to the present aspect, the falling risk information can be estimated according to the physical ability such as the grip strength, the dynamic balance, the lower limb muscle strength, the movement ability, and the static balance of the subject.

In an aspect of the present example embodiment, the physical ability estimation model outputs a grip strength value of the subject as the estimated value of the grip strength. The physical ability estimation model outputs, as the estimated value of the dynamic balance, a harmonic ratio of a waist in a moving direction, a vertical direction, and a left-right direction. The physical ability estimation model outputs a standing-up and sitting-down time in a chair stand-up test as the estimated value of the lower limb muscle strength. The physical ability estimation model outputs a TUG required time in a time up and go (TUG) test as the estimated value of the movement ability. The physical ability estimation model outputs a one-leg standing time in a one-leg standing test as the estimated value of the static balance. According to the present aspect, the falling risk suitable for the physical ability can be estimated by using the physical ability estimation model that outputs a specific estimated value related to the physical ability.

In an aspect of the present example embodiment, the falling risk estimation model is constructed by machine learning using, as the second feature amount, a principal component of which an index indicating a degree of separation of distribution between two groups into which subjects are classified depending on the subjects have experienced falling exceeds a predetermined value. The second estimation unit estimates the falling risk score by inputting the at least one second feature amount used for constructing the falling risk estimation model, among a plurality of second feature amounts constructed by the feature amount construction unit, to the falling risk estimation model. According to the present aspect, the falling risk can be estimated with higher accuracy by using the falling risk estimation model that has learned the second feature amount based on the index depending on the subjects have experienced falling.

In an aspect of the present example embodiment, the estimation device is mounted in a terminal device having a screen that can be visually recognized by the subject. For example, the estimation device displays falling risk information estimated using the first feature amount data related to the physical ability measured according to the gait of the subject and the attribute data of the subject on the screen of the terminal device. For example, the estimation device displays recommendation information according to the estimated falling risk information on the screen of the terminal device. For example, the estimation device displays, on the screen of the terminal device, a video related to training effective in reducing the falling risk as the recommendation information according to the falling risk information. According to the present aspect, by displaying the estimated falling risk information on the screen that can be visually recognized by the subject, the subject can check the information on his/her falling risk factor.

### Second Example Embodiment

Next, an estimation device according to a second example embodiment will be described with reference to the drawings. The estimation device according to the present example embodiment has a simplified configuration of the estimation device included in the estimation system according to the first example embodiment.

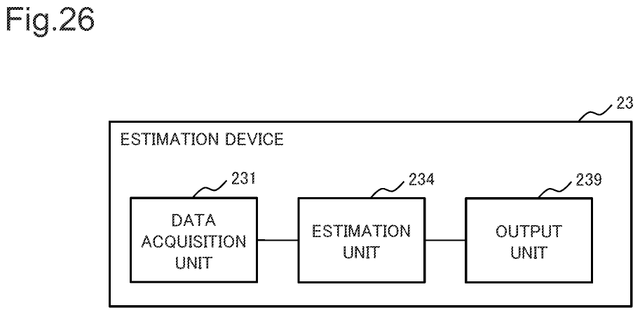

FIG. 26 is a block diagram illustrating an example of a configuration of an estimation device 23 according to the present example embodiment. The estimation device 23 includes a data acquisition unit 231, an estimation unit 234, and an output unit 239. The data acquisition unit 231 acquires first feature amount data related to a physical ability measured according to a gait of the subject and attribute data of the subject. The estimation unit 234 constructs second feature amount data related to a physical ability factor and an attribute factor by performing principal component analysis on the acquired first feature amount data and the acquired attribute data. The estimation unit 234 estimates falling risk information according to a falling risk factor using the constructed second feature amount data. The output unit 239 outputs the estimated falling risk information.

As described above, in the present example embodiment, the falling risk for the subject is estimated using the first feature amount data related to the physical ability measured according to the gait and the attribute data of the subject. That is, according to the present example embodiment, a falling risk factor can be estimated using the physical ability-related data measured according to the gait.

### (Hardware)

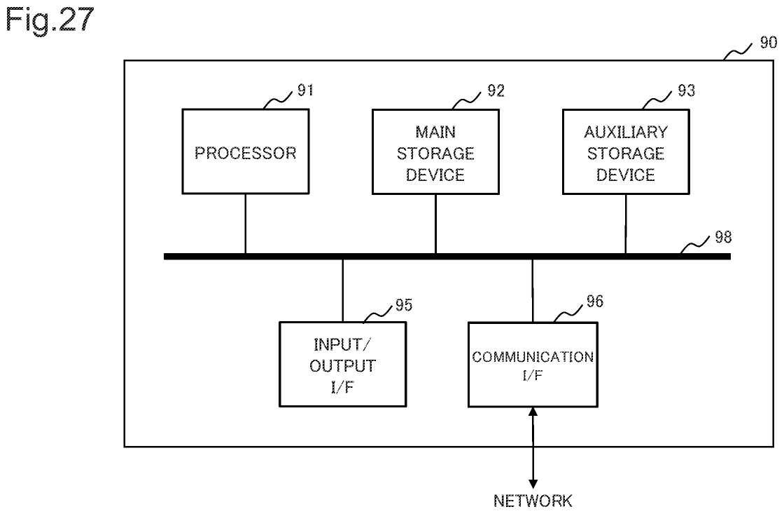

Here, a hardware configuration for executing the control or processing according to each of the above-described example embodiments of the present disclosure will be described using an information processing apparatus 90 illustrated in FIG. 27 as an example. Note that the information processing apparatus 90 of FIG. 27 is an example of the configuration for executing the control or processing according to each of the above-described example embodiments, and does not limit the scope of the present disclosure.

As illustrated in FIG. 27, the information processing apparatus 90 includes a processor 91, a main storage device 92, an auxiliary storage device 93, an input/output interface 95, and a communication interface 96. In FIG. 27, an interface is abbreviated as an I/F. The processor 91, the main storage device 92, the auxiliary storage device 93, the input/output interface 95, and the communication interface 96 are connected to each other via a bus 98 for data communication therebetween. In addition, the processor 91, the main storage device 92, the auxiliary storage device 93, and the input/output interface 95 are connected to a network such as the Internet or an intranet via the communication interface 96.

The processor 91 develops a program stored in the auxiliary storage device 93 or the like in the main storage device 92. The processor 91 executes the program developed in the main storage device 92. In the present example embodiment, a software program installed in the information processing apparatus 90 may be used. The processor 91 executes the control or processing according to each of the above-described example embodiments.

The main storage device 92 has an area in which a program is developed. A program stored in the auxiliary storage device 93 or the like is developed in the main storage device 92 by the processor 91. The main storage device 92 is achieved by, for example, a volatile memory such as a dynamic random access memory (DRAM). In addition, a nonvolatile memory such as a magnetoresistive random access memory (MRAM) may be included/added as the main storage device 92.

The auxiliary storage device 93 stores various data such as programs. The auxiliary storage device 93 is achieved by a local disk such as a hard disk or a flash memory. Note that various data may be stored in the main storage device 92, and the auxiliary storage device 93 may be omitted.

The input/output interface 95 is an interface for connecting the information processing apparatus 90 and a peripheral device to each other in accordance with a standard or a specification. The communication interface 96 is an interface for connection to an external system or device through a network such as the Internet or an intranet in accordance with a standard or a specification. The input/output interface 95 and the communication interface 96 may be constituted by a single interface connected to an external device.

An input device such as a keyboard, a mouse, or a touch panel may be connected to the information processing apparatus 90 if necessary. These input devices are used to input information and settings. In a case where the touch panel is used as an input device, a display screen of a display device may also serve as an interface of the input device. Data communication between the processor 91 and the input device may be mediated by the input/output interface 95.

Furthermore, the information processing apparatus 90 may include a display device for displaying information. In a case where the information processing apparatus 90 includes a display device, the information processing apparatus 90 preferably includes a display control device (not illustrated) for controlling the display of the display device. The display device may be connected to the information processing apparatus 90 via the input/output interface 95.

Furthermore, the information processing apparatus 90 may be equipped with a drive device. Between the processor 91 and the recording medium (program recording medium), the drive device mediates reading of data or a program from the recording medium, writing of a processing result of the information processing apparatus 90 to the recording medium, and the like. The drive device only needs to be connected to the information processing apparatus 90 via the input/output interface 95.

An example of the hardware configuration for enabling the control or processing according to each of the above-described example embodiments of the present disclosure has been described above. Note that the hardware configuration of FIG. 27 is an example of the hardware configuration for executing the control or processing according to each of the above-described example embodiments, and does not limit the scope of the present disclosure. In addition, a program for causing a computer to execute the control or processing according to each of the above-described example embodiments also falls within the scope of the present disclosure. Furthermore, a program recording medium recording the program according to each of the above-described example embodiments also falls within the scope of the present disclosure. The recording medium can be achieved by, for example, an optical recording medium such as a compact disc (CD) or a digital versatile disc (DVD). The recording medium may be achieved by a semiconductor recording medium such as a universal serial bus (USB) memory or a secure digital (SD) card. Furthermore, the recording medium may be achieved by a magnetic recording medium such as a flexible disk, or another recording medium. In a case where a program executed by the processor is recorded in the recording medium, the recording medium is a program recording medium.

The components of the above-described example embodiments may be combined in any manner. In addition, the components according to each of the above-described example embodiments may be achieved by software or by a circuit.

The previous description of embodiments is provided to enable a person skilled in the art to make and use the present invention. Moreover, various modifications to these example embodiments will be readily apparent to those skilled in the art, and the generic principles and specific examples defined herein may be applied to other embodiments without the use of inventive faculty. Therefore, the present invention is not intended to be limited to the example embodiments described herein but is to be accorded the widest scope as defined by the limitations of the claims and equivalents.

Further, it is noted that the inventor's intent is to retain all equivalents of the claimed invention even if the claims are amended during prosecution.

1. An estimation device comprising:
a first memory storing instructions; and
a first processor connected to the first memory and configured to execute the instructions to:
acquire first feature amount data related to a physical ability measured according to a gait of a subject and attribute data of the subject;
construct second feature amount data related to a physical ability factor and an attribute factor by performing principal component analysis on the acquired first feature amount data and the acquired attribute data,
estimate falling risk information according to a falling risk factor using the constructed second feature amount data; and
output the estimated falling risk information.

2. The estimation device according to claim 1, further comprising
a storage configured to store an estimation model that estimates at least one physical ability factor related to the falling risk factor as the first feature amount data and the attribute data are input, constructs a second feature amount by performing principal component analysis of the attribute factor included in the attribute data and the estimated physical ability factor, and outputs a falling risk score using the constructed second feature amount, wherein
the first processor is configured to execute the instructions to
input the acquired first feature amount data and the acquired attribute data to the estimation model, and
estimate the falling risk information using the at least one physical ability factor output from the estimation model.

3. The estimation device according to claim 2, wherein
the storage is configured to store
a physical ability estimation model that outputs the at least one physical ability factor as the first feature amount data and the attribute data are input,
a feature amount construction model that outputs at least one second feature amount by performing principal component analysis on the at least one attribute factor and the at least one physical ability factor as the at least one attribute factor and the at least one physical ability factor are input, and
a falling risk estimation model that outputs the falling risk score as the at least one second feature amount is input, and wherein
the first processor is configured to execute the instructions to
acquire the first feature amount data to be used in estimating the at least one physical ability factor related to the falling risk factor, the physical ability factor being extracted from gait waveform data generated using time-series data of sensor data measured according to the gait, and
input the first feature amount data and the attribute data to the physical ability estimation model, and estimates the at least one physical ability factor output from the physical ability estimation model as a physical ability of the subject,
construct the at least one second feature amount by inputting the at least one attribute factor and the at least one physical ability factor to the feature amount construction model, and
input the at least one second feature amount to the falling risk estimation model, and
estimate the falling risk information for the subject using the falling risk score output from the falling risk estimation model.

4. The estimation device according to claim 3, wherein
the attribute data includes a body mass index (BMI) and an age of the subject as the attribute factor.

5. The estimation device according to claim 4, wherein
the physical ability estimation model is configured to output, as the physical ability factor, estimated values related to a grip strength, a dynamic balance, a lower limb muscle strength, a movement ability, and a static balance as the attribute data and the first feature amount data are input.

6. The estimation device according to claim 5, wherein
the physical ability estimation model is configured to output
a grip strength value of the subject as the estimated value of the grip strength,
a harmonic ratio of a waist in a moving direction, a vertical direction, and a left-right direction as the estimated value of the dynamic balance,
a standing-up and sitting-down time in a chair stand-up test as the estimated value of the lower limb muscle strength,
a time up and go (TUG) required time in a TUG test as the estimated value of the movement ability, and
a one-leg standing time in a one-leg standing test as the estimated value of the static balance.

7. The estimation device according to claim 3, wherein
the falling risk estimation model is constructed by machine learning using, as the second feature amount, a principal component of which an index indicating a degree of separation of distribution between two groups into which subjects are classified depending on the subjects have experienced falling exceeds a predetermined value, and
the first processor is configured to execute the instructions to
estimate the falling risk score by inputting the at least one second feature amount used for constructing the falling risk estimation model, among a plurality of second feature amounts constructed by the feature amount construction means, to the falling risk estimation model.

8. The estimation device according to claim 1, wherein
the first processor is configured to execute the instructions to
output recommendation information for the subject to do decision making corresponding to the falling risk information.

9. An estimation system comprising:
the estimation device according to claim 1; and
a gait measurement device including
a sensor that measures a spatial acceleration and a spatial angular velocity, generates sensor data according to the gait using the measured spatial acceleration and spatial angular velocity, and outputs the generated sensor data,
a second memory storing instructions; and
a second processor connected to the second memory and configured to execute the instructions to
extract gait waveform data for one gait cycle from time-series data of the sensor data,
normalize the extracted gait waveform data,
extract a first feature amount to be used in estimating the falling risk factor from the normalized gait waveform data,
generate the first feature amount data including the extracted first feature amount, and
output the generated first feature amount data to the estimation device, wherein
the sensor is installed on footwear of a subject who is a target in estimating the falling risk factor.

10. An estimation method performed by a computer, the method comprising:
acquiring first feature amount data related to a physical ability measured according to a gait of a subject and attribute data of the subject;
constructing second feature amount data related to a physical ability factor and an attribute factor by performing principal component analysis on the acquired first feature amount data and the acquired attribute data;
estimating falling risk information according to a falling risk factor using the constructed second feature amount data; and
outputting the estimated falling risk information.

11. A non-transitory recording medium that records a program for causing a computer to execute:
acquiring first feature amount data related to a physical ability measured according to a gait of a subject and attribute data of the subject;
constructing second feature amount data related to a physical ability factor and an attribute factor by performing principal component analysis on the acquired first feature amount data and the acquired attribute data;
estimating falling risk information according to a falling risk factor using the constructed second feature amount data; and
outputting the estimated falling risk information.
